(19) United States
(12) Patent Application Publication
(10) Pub. No.: US 2023/0346939 A1
(43) Pub. Date: Nov. 2, 2023

(54) PROGRAMMING OF REGULATORY T CELLS BY EXTRACELLULAR VESICLES

(71) Applicant: City of Hope, Duarte, CA (US)

(72) Inventor: Fouad Kandeel, Duarte, CA (US)

(21) Appl. No.: 18/026,336

(22) PCT Filed: Sep. 15, 2021
(86) PCT No.: PCT/US2021/050487

§ 371 Date: Mar. 14, 2023

Related U.S. Application Data
(60) Provisional application No. 63/078,742, filed on Sep. 15, 2020.

(51) Int. Cl. A61K 39/00; C12N 5/0783; C07K 14/725; C07K 16/28; C07K 14/73

(52) U.S. Cl. CPC A61K 39/46433; C12N 5/0637; C07K 14/7051; C07K 16/2896; C07K 14/70514; A61K 39/4611; A61K 39/4621; A61K 39/4631; A61K 39/46434; C12N 2502/1121; C12N 2501/231; C12N 2501/2302; C07K 2317/622; C07K 2317/24; C07K 2319/03; C07K 2319/61; A61K 2239/15; A61K 2239/21; A61K 2239/26

(57) ABSTRACT

Provided herein, inter alia, are compositions and methods for reprogramming immune cells for treating or preventing immune disorders. The methods include contacting immune cells with antigens, and administering the resultant immune cells to a subject who has an immune disorder.

### CROSS-REFERENCE TO RELATED APPLICATIONS

This application claims priority to U.S. Provisional Application No. 63/078,742, filed Sep. 15, 2020, which is hereby incorporated by reference in its entirety and for all purposes.

### BACKGROUND OF THE INVENTION

There has been massive investment on the development of successful immune modulation strategies to curb the progression, prevent and treat the development of Type 1 diabetes (T1D) by numerous investigative groups, with little or no success. Although polyclonal regulatory T cells (Tregs) have been successfully expanded, the clinical utility of these cells has so far yielded suboptimal therapeutic results. Currently, there is no viable antigen-specific Treg product that has shown successful therapeutic effects in T1D patients. Previous preclinical, and early phase 2 clinical studies using non-specific, polyclonal Tregs did not demonstrate C-peptide preservation in newly diagnosed T1D patients (NCT02691247). Successful development of a diabetes antigen-specific Treg population will better direct these cells to the islet microenvironment and thereby enhance their therapeutic potential to protect beta cells from autoimmune-induced damage. Provided herein, inter alia, are solutions to these and other problems in the art.

### BRIEF SUMMARY OF THE INVENTION

In an aspect is provided a method of generating a reprogrammed T regulatory (Treg) cell, the method including: (a) contacting a dendritic cell (DC) with interleukin-10 (IL-10), thereby producing a tolerogenic dendritic cell (tolDC); and (b) contacting the tolDC with an islet extracellular vesicle (EV), thereby producing an antigen-loaded tolDC; and contacting a Treg cell with the antigen-loaded tolDC, thereby producing the reprogrammed Treg cell.

In another aspect a reprogrammed Treg cell made by a method provided herein including embodiments thereof is provided.

In another aspect a method of treating Type 1 diabetes in a subject in need thereof is provided, the method including administering to the subject a therapeutically effective amount of a reprogrammed Treg provided herein including embodiments thereof.

In another aspect a method of treating or preventing Type 1 diabetes in a subject in need thereof is provided, the method including: (a) contacting a tolerogenic dendritic cell (tolDC) with an islet extracellular vesicle (EV), thereby producing an antigen-loaded tolDC; (b) contacting a T regulatory (Treg) cell with the antigen-loaded tolDC, thereby generating a reprogrammed Treg cell; and (c) administering a therapeutically effective amount of the reprogrammed Treg cell to the subject, thereby treating or preventing Type 1 diabetes in the subject.

In an aspect is provided a method of generating tolerogenic dendritic cells (tolDCs) including contacting extracellular vesicles from islet cells (islet-EV) with CD14+ monocytes, wherein the contacting results in tolDCs.

In an aspect is provided a tolerogenic dendritic cell generated by any of the methods provided herein including embodiments thereof.

In another aspect a method of generating reprogrammed T-regulatory cells (Tregs) is provided, the method including contacting isolated polyclonal Tregs with the tolerogenic dendritic cells provided herein including embodiments thereof, wherein the contacting results in Tregs expressing CD4+, CD25+ and FoxP3.

In an aspect is provided a reprogrammed Treg generated by the methods provided herein, including embodiments thereof.

In an aspect is provided a method of treating Type 1 diabetes to a subject in need, the method including administering the reprogrammed Treg provided herein, including embodiments thereof.

In an aspect is provided a method of treating or preventing type 1 diabetes in a subject in need thereof, the method including administering to the subject a therapeutically effective amount of an autologous regulatory T cell; thereby treating or preventing the progression of type 1 diabetes in the subject.

### BRIEF DESCRIPTION OF THE DRAWINGS

FIG. 1 is a schematic diagram showing an embodiment of a method of education and expansion of aTregs with tolDC and modification of the aTregs to express a binding domain to GPR44. Bio-distribution studies are performed to verify the migration of GPR44-CAR-aTregs to the islet environment in a mice mode. Targets in addition to GPR44 include GLP-1R, FXYD2, and NTPDase3.

FIG. 2A is a graph showing that expanded Tregs from T1D donor prolonged mouse survival (triangle) in comparison to a control no-treatment group (square) and immune suppressants alone (circle). Immune suppressants tested include ATG, GM-CSF, and sirolimus.

FIG. 2B is a graph illustrating polyclonal Treg delay/prevent the development of diabetes in the autoimmune NOD mouse model. Diabetes develops in NOD mice by age 18 weeks (circle). The onset of autoimmune diabetes in this model can be delayed/prevented by the treatment with Tregs. Sirolimus alone (square), had little effect but may augment the effect of Tregs (diamond).

FIG. 3 is a representative image of an immunoblot gel showing autoantigens detected in islet EV. Autoantigens, GAD65 and IA2 are detected on islet EVs from three healthy donors. EV marker CD81 is detected in both serum and islet EVs.

FIG. 4A illustrates that DC-10 shows superior tolerogenic features. FACS analysis shows that DC-10 expressed lower levels of CD86, HLA-DR, CD1a, CD80, CD83, CD40, and higher levels of CD14, JILT-3 than mDC.

FIG. 4B illustrates that DC-10 (tolerogenic) secrete higher levels of IL-10, TGF-β, and IL-6 but decreased levels of IL-1beta, TNF-a, and IL-23.

FIG. 5 illustrates Foxp3 expression on expanded aTreg by FACS analysis. Both polyclonal Tregs (nTregs) shown in the upper panels and aTregs, shown in the bottom panels, expressed a high % of Foxp3.

FIG. 6 are bar graphs showing aTregs secrete higher immunosuppressive cytokine levels. aTregs and polyclonal Tregs (nTregs) were re-stimulated after the expansion phase. The culture supernatant was assayed for cytokines analysis and showed higher IL-10 and TGF-b secretion than that of the polyclonal Tregs (nTregs).

FIG. 7 are bar graphs showing aTregs inhibit Teff proliferation. Co-culturing of aTregs or nTreg with PBMC in the presence of CD3/CD28 beads demonstrate that aTregs suppress CD4⁺ and CD8⁺ T cell proliferation more efficiently than polyclonal Tregs (nTregs).

FIG. 8 is a schematic of the flow process of aTreg generation. After leukaphoresis from T1D patient, CD14 cells are isolated and induced to tolDC. After tolDCs are pulsed with islet EV antigens, Tregs are educated for two rounds of reprogramming and expanded for 1-week post-education, which then may be followed by functional assays.

FIG. 9 is a schematic description of GPR44-CAR-aTreg production, including the selection of aTregs and lentiviral transduction.

### DETAILED DESCRIPTION OF THE INVENTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.
The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or sub-value there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages.

Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids.

Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The terms “peptidyl” and “peptidyl moiety” refer to a peptide attached to the remainder of the molecule (e.g., the recombinant protein provided herein or the peptide domain forming part of the recombinant protein provided herein). A peptidyl moiety may be substituted with a chemical linker that serves to attach the peptidyl moiety to the remainder of the recombinant protein (e.g., the transmembrane domain, the spacer region or the peptidyl linker). The peptidyl moiety may also be substituted with additional chemical moieties (e.g., additional R substituents).

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990)J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen-binding, specificity, etc.).

Antibodies are large, complex molecules (molecular weight of ˜150,000 Da or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site (paratope), which docks onto the target antigen (epitope). The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific diabodies, trispecifictriabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see Fundamental Immunology (Paul ed., 3rd ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A “humanized antibody” is a genetically engineered antibody in which at least one CDR (or functional fragment thereof) from a mouse antibody (“donor antibody,” which can also be rat, hamster or other non-human species) are grafted onto a human antibody (“acceptor antibody”). In embodiments, more than one mouse CDR is grafted (e.g., all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR or (2) the amino acid is in the human framework region (e.g., the amino acid is immediately adjacent to one of the CDRs). See, U.S. Pat. Nos. 5,530,101 and 5,585,089, incorporated herein by reference, which provide detailed instructions for construction of humanized antibodies. Although humanized antibodies often incorporate all six CDRs (e.g., as defined by Kabat, but often also including hypervariable loop H1 as defined by Chothia) from a mouse antibody, they can also be made with fewer mouse CDRs and/or less than the complete mouse CDR sequence (e.g., a functional fragment of a CDR) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al., Journal of Immunology, 164:1432-1441, 2000).

The term “bispecific antibody” as provided herein is used according to its conventional meaning well known in the art and refers to a bispecific recombinant protein capable to bind to two different antigens. The bispecific antibody may bind two antigens simultaneously. In contrast to traditional monoclonal antibodies, BiTE antibodies consist of two independently different antibody regions (e.g., two single-chain variable fragments (scFv)), each of which binds a different antigen. One antibody region may engage effector cells (e.g., T cells) by binding an effector cell-specific antigen (e.g., CD4 molecule) and the second antibody region may bind a target cell (e.g., beta cell) through a cell surface antigen (e.g., GPR44) expressed by said target cell. Binding of the bispecific antibody to the two antigens will link the effector cell (e.g., T cell) to the target cell (e.g., beta cell). The effector cell (e.g., T cell) may then be activated via effector cell-specific antigen signaling.

As used herein, the terms “isolated”, “isolating”, “purified” refer to cells or molecules that have been separated from their natural milieu or from components of the environment in which they are produced. Thus, “isolated” or “isolating” do not necessarily refer to the degree of purity of a cell or molecule of the present invention. For example, a naturally occurring cell or molecule (e.g., a Treg cell, an APC, protein etc.) present in a living animal, including humans, is not isolated. However, the same cell, or molecule, separated from some or all of the coexisting materials in the animal, is considered isolated. As a further example, cells that are present in a sample of blood obtained from a person would be considered isolated. It should be appreciated that cells obtained from such a sample using further purification steps would also be referred to as isolated, in keeping with the notion that isolated does not refer to the degree of purity of the cells.

A “label”, “detectable domain” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. In embodiments, contacting a cell (e.g. Treg cell) with another cell (e.g. tolDC) refers to growing the cells in the same cell culture.

The term “contacting” may include allowing two species to react, interact, or physically touch, wherein the two species may be a cell as described herein and an extracellular vesicle. In some embodiments, contacting refers to allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The terms “agonist”, “activator”, “upregulator”, etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor”, “repressor”, “antagonist”, or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease means that the disease is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, the biological sample includes beta cells.

As used herein, the term “allogeneic transplant” or “allogeneic transfusion” refers to the transfer of biological material (e.g. tissue or blood) to a recipient from a genetically non-identical donor of the same species. The term “transplant” may be referred to as an allograft, allogeneic transplant, or homograft. For example, the allogeneic transplant may be a tissue transplant or organ transplant. Thus, an allogeneic transplant may include transfer of tissue, a group of cells or an organ to a recipient that is genetically non-identical to the donor. In embodiment, the tissue transplant includes stem cells. In embodiments, the tissue transplant includes islet cells. In embodiments, the tissue transplant includes immune cells (e.g. Treg cells).

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “beta cell” or “3 cell” is a type of cell found in pancreatic islets. Normal functioning beta cells synthesize and secrete insulin and amylin. In type 1 diabetes (T1DM), beta cell function and mass are decreased, primarily due to the autoimmune attack of auto reactive T cells against islet beta cells.

The term “pancreatic progenitor”, “pancreatic precursor,” or “progenitor pancreatic cell” are used interchangeably herein and refer to a stem cell which are less differentiated than pancreatic endocrine progenitor cells, and are capable of forming all cell types of the pancreatic lineage, including pancreatic endocrine cells, pancreatic exocrine cells or pancreatic duct cells. Under the right conditions, they can form the subset of pancreatic endocrine cells, e.g. beta cells that produce insulin; alpha cells that produce glucagon; delta cells (or D cells) that produce somatostatin; and/or PP cells that produce pancreatic polypeptide.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The terms “disease” or “condition” refer to a state of being or health status of a patient or subject capable of being treated with a compound, pharmaceutical composition, or method provided herein. The terms “disease” or “condition” refer to a state of being or health status of a patient or subject capable of being treated with a compound, pharmaceutical composition, or method provided herein.

As used herein, the term “autoimmune disease” refers to a disease or condition in which a subject's immune system has an aberrant immune response against a substance that does not normally elicit an immune response in a healthy subject. An autoimmune disease typically arises from altered immune reactions against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, and allergic asthma.

The term “diabetes” or “diabetic disorder” or “diabetes mellitus,” as used interchangeably herein, refers to a disease which is marked by elevated levels of sugar (glucose) in the blood. Diabetes can be caused by too little insulin (a protein produced by the pancreas to regulate blood sugar), resistance to insulin, or both. Diabetes mellitus includes, without limitation, type 1 diabetes, type 2 diabetes, or surgical diabetes.

The term “type 1 diabetes,” as used herein, refers to a chronic disease that occurs when the pancreas produces too little insulin to regulate blood sugar levels appropriately. Type 1 diabetes is also interchangeably referred to as “insulin-dependent diabetes mellitus,” “IDMM,” “juvenile onset diabetes,” “autoimmune diabetes,” and “diabetes-type 1.” Type 1 diabetes is the result of a progressive autoimmune destruction of the pancreatic 3-cells with subsequent insulin deficiency.

The term “associated” or “associated with” in the context of a substance or substance activity or function associated with a disease (e.g., autoimmune disease (e.g. type 1 diabetes)) means that the disease (e.g. cancer, autoimmune disease (e.g. type 1 diabetes)) is caused by (in whole or in part), or a symptom of the disease is caused by (in whole or in part) the substance or substance activity or function.

The terms “treating” or “treatment” refer to any indicia of success in the therapy or amelioration of an injury, disease (e.g. T1D), pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. In embodiments, treating refers to treating a subject having a disease.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent (e.g. reprogrammed Treg cell). The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of a disease (e.g. T1D) or disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease (e.g. T1D) or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human.

As used herein, the term “donor” refers to a subject who provides an organ, tissue, or group of cells for transplantation to a recipient. In embodiments, the donor may be the same subject as the recipient (e.g. autotransplantation, autologous regulatory T cells, etc.). In embodiments, the donor may be a different subject than the recipient (e.g. allotransplant, heterologous regulatory T cells, etc.). For example, a donor may provide regulatory T cells, wherein the regulatory T cells will be administered to a subject who is different than the donor. For example, a donor may provide extracellular vesicles from pancreatic islets, wherein a product made using the extracellular vesicles (e.g. reprogrammed Treg cell) will be administered to a subject who is different than the donor. In embodiments, a donor is healthy, e.g. does not suffer from Type I diabetes.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. In embodiments, the dosage of a cell therapy can be estimated initially from preclinical data comparing the relative potency of a formulation with the standard formulation in animal studies. For example, a minimal cell dose to achieve diabetes remission in animal studies can be compared between formulations. In embodiments, in dose-finding studies, C-peptide (a non-metabolized byproduct of insulin production) concentration at baseline and after administration of glucose can also be measured and compared between the old and new formulation. Such information can be used to more accurately determine useful doses in humans. Levels of C-peptide in plasma may be measured, for example, using enzyme-linked immunosorbent assay.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as aerosol, dry powder, nasal spray, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be co-administered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g. rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powders, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J Microencapsul. 13:293-306, 1996; Chonn, Curr. Opin. Biotechnol. 6:698-708, 1995; Ostro, Am. J. Hosp. Pharm. 46:1576-1587, 1989).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a reprogrammed Treg cell compared to the activity of a polyclonal Treg cell as described herein (including embodiments and examples).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

The term “expand” as used herein refers to increasing the number of cells in a cell culture. The culture medium may include growth factors, serum, cytokines or and other additives to help cells grow and/or differentiate.

Thus, “expanding regulatory T cells” or “expanding Tregs” refers to the process of proliferating Tregs in a cell culture, thereby forming a population of “expanded regulatory T cells” or “expanded Tregs”. In embodiments, the Tregs are expanded 1.5 fold to more than 100 fold compared to the original number or concentration of Tregs. Tregs may be expanded in the presence of one or more agents that assist in the proliferation or survival of the cells. The agent may be a drug, antibody, cell, or cytokine. The agent may be, for example, rapamycin, antigen presenting cell (APC), interleukin 2 (IL-2), interleukin 15 (IL-15), CD3/CD28 antibody, or a combination thereof.

The term “T-cell surface glycoprotein CD8” or “CD8” as referred to herein includes any of the recombinant or naturally-occurring forms of the T-cell surface glycoprotein CD8 (CD8) protein, also known as cluster of differentiation 8, or variants or homologs (including functional fragments thereof) thereof that maintain CD8 protein activity (e.g. within at least 50%, 80%, 90%, 95%, 96%, 97%, 98%, 99% or 100% activity compared to CD8 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 protein. In embodiments, the CD8 protein is substantially identical to the protein identified by the UniProt reference number P10966 or a variant or homolog having substantial identity thereto. In embodiments, the CD8 protein is substantially identical to the protein identified by the UniProt reference number P01732 or a variant or homolog having substantial identity thereto.

The term “T-cell surface glycoprotein CD4” or “CD4” as referred to herein includes any of the recombinant or naturally-occurring forms of the T-cell surface glycoprotein CD4 (CD4) protein, also known as T-cell surface antigen T4/Leu-3, cluster of differentiation 4, or variants or homologs thereof (including functional fragments thereof) that maintain CD4 protein activity (e.g. within at least 50%, 80%, 90%, 95%, 96%, 97%, 98%, 99% or 100% activity compared to CD4 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 protein. In embodiments, the CD4 protein is substantially identical to the protein identified by the UniProt reference number P01730 or a variant or homolog having substantial identity thereto.

The term “monocyte differentiation antigen CD14” or “CD14” as referred to herein includes any of the recombinant or naturally-occurring forms of the monocyte differentiation antigen CD14 (CD14) protein, also known as Myeloid cell-specific leucine-rich glycoprotein, cluster of differentiation 14, or variants or homologs thereof (including functional fragments thereof) that maintain CD14 protein activity (e.g. within at least 50%, 80%, 90%, 95%, 96%, 97%, 98%, 99% or 100% activity compared to CD14 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD14 protein. In embodiments, the CD14 protein is substantially identical to the protein identified by the UniProt reference number P08571 or a variant or homolog having substantial identity thereto.

The term “T-cell-specific surface glycoprotein CD28” or “CD28” as referred to herein includes any of the recombinant or naturally-occurring forms of the T-cell-specific surface glycoprotein CD28 (CD28) protein, also known as TP44, cluster of differentiation 28, or variants or homologs thereof (including functional fragments thereof) that maintain CD28 protein activity (e.g. within at least 50%, 80%, 90%, 95%, 96%, 97%, 98%, 99% or 100% activity compared to CD28 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “T-lymphocyte activation antigen CD80” or “CD80” as referred to herein includes any of the recombinant or naturally-occurring forms of the T-lymphocyte activation antigen CD80 (CD80) protein, also known as Activation B7-1 antigen, BB1, CTLA-4 counter-receptor B7.1, cluster of differentiation 80, or variants or homologs thereof (including functional fragments thereof) that maintain CD80 protein activity (e.g. within at least 50%, 80%, 90%, 95%, 96%, 97%, 98%, 99% or 100% activity compared to CD80 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD80 protein. In embodiments, the CD80 protein is substantially identical to the protein identified by the UniProt reference number P33681 or a variant or homolog having substantial identity thereto.

The term “T-lymphocyte activation antigen CD86” or “CD86” as referred to herein includes any of the recombinant or naturally-occurring forms of the T-lymphocyte activation antigen CD86 (CD86) protein, also known as Activation B7-2 antigen, B70, CTLA-4 counter-receptor B7.2, cluster of differentiation 86, or variants or homologs thereof (including functional fragments thereof) that maintain CD86 protein activity (e.g. within at least 50%, 80%, 90%, 95%, 96%, 97%, 98%, 99% or 100% activity compared to CD86 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD86 protein. In embodiments, the CD86 protein is substantially identical to the protein identified by the UniProt reference number P42081 or a variant or homolog having substantial identity thereto.

A “interleukin-10 protein” or “IL-10” as referred to herein includes any of the recombinant or naturally-occurring forms of the interleukin-10 (IL-10) also known as human cytokine synthesis inhibitory factor (CSIF) or variants or homologs thereof (including functional fragments thereof) that maintain IL-10 protein activity (e.g. within at least 50%, 80%, 90%, 95%, 96%, 97%, 98%, 99% or 100% activity compared to IL-10 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-10 protein. In embodiments, the IL-10 protein is substantially identical to the protein identified by the UniProt reference number P22301 or a variant or homolog having substantial identity thereto.

A “Forkhead box protein P3” or “FoxP3” as referred to herein includes any of the recombinant or naturally-occurring forms of the Forkhead box protein P3 (FoxP3) also known as scurfin, or variants or homologs thereof (including functional fragments thereof) that maintain FoxP3 protein activity (e.g. within at least 50%, 80%, 90%, 95%, 96%, 97%, 98%, 99% or 100% activity compared to FoxP3 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FoxP3 protein. In embodiments, the FoxP3 protein is substantially identical to the protein identified by the UniProt reference number Q9BZS1 or a variant or homolog having substantial identity thereto.

A “Interleukin-2 receptor alpha chain protein” or “CD25” as referred to herein includes any of the recombinant or naturally-occurring forms of the Interleukin-2 receptor alpha chain protein (CD25) also known as Interleukin-2 receptor subunit alpha, or variants or homologs thereof (including functional fragments thereof) that maintain CD25 protein activity (e.g. within at least 50%, 80%, 90%, 95%, 96%, 97%, 98%, 99% or 100% activity compared to CD25 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD25 protein. In embodiments, the CD25 protein is substantially identical to the protein identified by the UniProt reference number P01589 or a variant or homolog having substantial identity thereto.

A “Interleukin-4 protein” or “IL-4” as referred to herein includes any of the recombinant or naturally-occurring forms of the Interleukin-4 (IL-4) also known as B-cell stimulatory factor 1, Lymphocyte stimulatory factor 1, or variants or homologs thereof (including functional fragments thereof) that maintain IL-4 protein activity (e.g. within at least 50%, 80%, 90%, 95%, 96%, 97%, 98%, 99% or 100% activity compared to IL-4 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-4 protein. In embodiments, the IL-4 protein is substantially identical to the protein identified by the UniProt reference number P05112 or a variant or homolog having substantial identity thereto.

A “Tumor necrosis factor alpha protein” or “TNFα” as referred to herein includes any of the recombinant or naturally-occurring forms of the Tumor necrosis factor alpha protein (TNFα) also known as cachexin, Tumor necrosis factor, or variants or homologs thereof (including functional fragments thereof) that maintain TNFα protein activity (e.g. within at least 50%, 80%, 90%, 95%, 96%, 97%, 98%, 99% or 100% activity compared to TNFα protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TNFα protein. In embodiments, the TNFα protein is substantially identical to the protein identified by the UniProt reference number P01375 or a variant or homolog having substantial identity thereto.

A “Interferon-gamma protein” or “IFN-gamma” as referred to herein includes any of the recombinant or naturally-occurring forms of the Interferon-gamma protein (IFN-gamma) also known as Immune interferon, or variants or homologs thereof (including functional fragments thereof) that maintain IFN-gamma protein activity (e.g. within at least 50%, 80%, 90%, 95%, 96%, 97%, 98%, 99% or 100% activity compared to IFN-gamma protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IFN-gamma protein. In embodiments, the IFN-gamma protein is substantially identical to the protein identified by the UniProt reference number P01579 or a variant or homolog having substantial identity thereto.

A “Sodium/potassium-transporting ATPase gamma chain protein” or “FXYD2” as referred to herein includes any of the recombinant or naturally-occurring forms of the Sodium/potassium-transporting ATPase gamma chain protein (FXYD2) also known as FXYD domain-containing ion transport regulator 2, Sodium pump gamma chain or variants or homologs thereof (including functional fragments thereof) that maintain FXYD2 protein activity (e.g. within at least 50%, 80%, 90%, 95%, 96%, 97%, 98%, 99% or 100% activity compared to FXYD2 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FXYD2 protein. In embodiments, the FXYD2 protein is substantially identical to the protein identified by the UniProt reference number P54710 or a variant or homolog having substantial identity thereto.

A “Prostaglandin DP₂ receptor protein” or “GPR44” as referred to herein includes any of the recombinant or naturally-occurring forms of the Prostaglandin DP₂ receptor protein (GPR44) also known as Chemoattractant receptor-homologous molecule expressed on Th2 cells, G-protein coupled receptor 44, CD294 or variants or homologs thereof (including functional fragments thereof) that maintain GPR44 protein activity (e.g. within at least 50%, 80%, 90%, 95%, 96%, 97%, 98%, 99% or 100% activity compared to GPR44 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GPR44 protein. In embodiments, the GPR44 protein is substantially identical to the protein identified by the UniProt reference number Q9Y5Y4 or a variant or homolog having substantial identity thereto.

A “Glucagon-like peptide 1 receptor” or “GLP-1R” as referred to herein includes any of the recombinant or naturally-occurring forms of the Glucagon-like peptide 1 receptor (GLP-1R) also known as a receptor for glucagon-like peptide 1 (GLP-1) hormone or variants or homologs thereof (including functional fragments thereof) that maintain GLP-1R protein activity (e.g. within at least 50%, 80%, 90%, 95%, 96%, 97%, 98%, 99% or 100% activity compared to GLP-1R protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GLP-1R protein. In embodiments, the GLP-1R protein is substantially identical to the protein identified by the UniProt reference number P43220 or a variant or homolog having substantial identity thereto.

A “transforming growth factor beta protein” or “TGF-beta” as referred to herein includes any of the recombinant or naturally-occurring forms of the transforming growth factor beta protein (TGF-beta) also known as Cetermin, Glioblastoma-derived T-cell suppressor factor, or variants or homologs thereof (including functional fragments thereof) that maintain TGF-beta protein activity (e.g. within at least 50%, 80%, 90%, 95%, 96%, 97%, 98%, 99% or 100% activity compared to TGF-beta protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TGF-beta protein. In embodiments, the TGF-beta protein is substantially identical to the protein identified by the UniProt reference number P61812 or a variant or homolog having substantial identity thereto. In embodiments, the TGF-beta protein is substantially identical to the protein identified by the UniProt reference number P01137 or a variant or homolog having substantial identity thereto. In embodiments, the TGF-beta protein is substantially identical to the protein identified by the UniProt reference number P10600 or a variant or homolog having substantial identity thereto.

A “Granulocyte-macrophage colony-stimulating factor protein” or “GM-CSF” as referred to herein includes any of the recombinant or naturally-occurring forms of the Granulocyte-macrophage colony-stimulating factor protein (GM-CSF) also known as CSF2, or variants or homologs thereof (including functional fragments thereof) that maintain GM-CSF protein activity (e.g. within at least 50%, 80%, 90%, 95%, 96%, 97%, 98%, 99% or 100% activity compared to GM-CSF protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GM-CSF protein. In embodiments, the GM-CSF protein is substantially identical to the protein identified by the UniProt reference number P04141 or a variant or homolog having substantial identity thereto. In embodiments, the GM-CSF protein is substantially identical to the protein identified by the UniProt reference number P04141 or a variant or homolog having substantial identity thereto.

A “T-cell surface glycoprotein CD1a protein” or “CD1a” as referred to herein includes any of the recombinant or naturally-occurring forms of the T-cell surface glycoprotein CD1a also known as T-cell surface antigen T6/Leu-6, or variants or homologs thereof (including functional fragments thereof) that maintain CD1a protein activity (e.g. within at least 50%, 80%, 90%, 95%, 96%, 97%, 98%, 99% or 100% activity compared to CD1a protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD1a protein. In embodiments, the CD1a protein is substantially identical to the protein identified by the UniProt reference number P06126 or a variant or homolog having substantial identity thereto. In embodiments, the CD1a protein is substantially identical to the protein identified by the UniProt reference number P06126 or a variant or homolog having substantial identity thereto.

A “Tumor necrosis factor receptor superfamily member 5” or “CD40” as referred to herein includes any of the recombinant or naturally-occurring forms of the Tumor necrosis factor receptor superfamily member 5, or CD40 also known as B-cell surface antigen CD40, Bp50, CD40L receptor, or variants or homologs thereof (including functional fragments thereof) that maintain CD40 protein activity (e.g. within at least 50%, 80%, 90%, 95%, 96%, 97%, 98%, 99% or 100% activity compared to CD40 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD40 protein. In embodiments, the CD40 protein is substantially identical to the protein identified by the UniProt reference number P25942 or a variant or homolog having substantial identity thereto. In embodiments, the CD40 protein is substantially identical to the protein identified by the UniProt reference number P25942 or a variant or homolog having substantial identity thereto.

A “Leukocyte immunoglobulin-like receptor subfamily B member 4 protein” or “ILT-3” as referred to herein includes any of the recombinant or naturally-occurring forms of the Leukocyte immunoglobulin-like receptor subfamily B member 4 protein ILT-3 also known as CD85 antigen-like family member K, Leukocyte immunoglobulin-like receptor 5, Monocyte inhibitory receptor HM18 expressed on monocytes, macrophages and DC, or variants or homologs thereof (including functional fragments thereof) that maintain ILT-3 protein activity (e.g. within at least 50%, 80%, 90%, 95%, 96%, 97%, 98%, 99% or 100% activity compared to ILT-3 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ILT-3 protein. In embodiments, the ILT-3 protein is substantially identical to the protein identified by the UniProt reference number Q8NHJ6 or a variant or homolog having substantial identity thereto. In embodiments, the ILT-3 protein is substantially identical to the protein identified by the UniProt reference number Q8NHJ6 or a variant or homolog having substantial identity thereto.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

The term “immunosuppressive cytokine” refers to immunoregulatory molecules that are negative mediators of inflammation or the immune response. Immunosuppressive cytokines may down regulate the production of pro-inflammatory cytokines, and inhibit T cell proliferation, activation and/or differentiation. In embodiments, immunosuppressive cytokines include interleukin (IL)-1 receptor antagonist, IL-4, IL-6, IL-10 IL-11, and IL-13. In certain circumstances, immunosuppressive cytokines may be pro-inflammatory cytokines. Dependent on circumstances, Leukemia inhibitory factor, interferon-alpha, IL-6, and transforming growth factor (TGF)-β may function as either anti-inflammatory or pro-inflammatory cytokines.

The term “pro-inflammatory cytokine” refers to signaling molecules (e.g. cytokines) that are positive mediators of inflammation or the immune response. Pro-inflammatory cytokines may recruit immune cells (e.g. leukocytes) to a target site, promote proliferation, activation and/or differentiation of T cells, or upregulate expression and/or release of pro-inflammatory enzymes. In embodiments, pro-inflammatory cytokines decrease production of immunosuppressive cytokines. Pro-inflammatory cytokines may be secreted from immune cells like helper T cells (Tₕ) and macrophages, and certain other cell types that promote inflammation. In embodiments, pro-inflammatory cytokines include IL-1, IL-6, IL-8, IL-12, IL-18, tumor necrosis factor alpha (TNF-α), interferon gamma (IFNγ), and granulocyte-macrophage colony stimulating factor (GM-CSF).

The term “tolerogenic” refers generally to immune-suppressive properties, or the ability to maintain a state of non-responsiveness of the immune system. For example, tolerogenic therapies aim to induce immune tolerance by using either inherent tolerance mechanisms or by inducing tolerance mechanisms in certain immune cells. A tolerogenic immune cell may refer to an immune cell that may decrease proliferation of effector T cells (e.g. cytotoxic T cells, CT Ls, T-killer cells, killer T cells, etc.), increase production of immunosuppressive cytokines, decrease production of proinflammatory cytokines, or decrease cell lysis. In embodiments, immunosuppressiveness or non-responsiveness of the immune system is one or more of decreased production or activation of a transcription factor (e.g. NF-κB, C/EBP-β, etc.) that results in production or activation of a proinflammatory cytokine.

“Tolerogenic dendritic cell”, or “tolDC” refers to dendritic cells that use immunosuppressive mechanisms to induce tolerance to self and/or foreign antigens. In embodiments, tolerogenic DCs maintain central and peripheral tolerance through induction of T cell clonal deletion, T cell anergy and generation and activation of regulatory T (Treg) cells. Immunosuppressive mechanisms include one or more of reduced T cell proliferation, Treg differentiation, and suppression of effector T cell proliferation and function. TolDCs are typically characterized by an immature phenotype with low expression levels of co-stimulatory and MHC molecules, in addition to altered cytokine production. TolDCs may secrete anti-inflammatory cytokines such as interleukin-10 (IL-10) and transforming growth factor-β. Tolerogenic DCs may further express different inhibitory receptors, for example, programmed cell death ligand (PDL)-1, PDL-2, inhibitory Ig-like transcripts (TLT), and cytotoxic T-lymphocyte-associated protein 4 (CTLA-4). Methods for generating tolDC are known in the art and are described in more detail in Gregori, S. et al. Differentiation of type 1 T regulatory cells (Tr1) by tolerogenic DC-10 requires the IL-10-dependent ILT4/HLA-G pathway; Blood, 12 Aug. 2010, Volume 116, No. 6; DOI 10.1182/blood-2009-07-234872; and Raker, V. K. Tolerogenic Dendritic Cells for Regulatory T Cell induction in Man; Front. Immunol, November 1015, Volume 6, Article 569; doi: 10.3389/fimmu.2015.00569; which are incorporated by reference herein in their entirety and for all purposes.

“Extracellular vesicle” or “EV” as used herein refers to heterogeneous membrane-enclosed particles that are released from a cell that carry a cargo of one or more of proteins, nucleic acids, lipids, metabolites, or organelles from the parent cell. Thus, “islet extracellular vesicles” are EVs that are produced by pancreatic islets or beta cells. Islet EVs include proteins, nucleic acids, and/or lipids from pancreatic islets and beta cells. Thus, in embodiments, islet extracellular vesicles include antigens (e.g. autoantigens) which may induce production of cytokines, which may modulate an immune response. In autoimmune disorders (e.g. Type I diabetes) islet EVs may include pro-inflammatory cytokines that stimulate immune cells (e.g. dendritic cells) to activate an immune response.

The term “heterologous extracellular vesicle” or “heterologous EV” as used herein refers to extracellular vesicles derived from an individual that is typically only the donor of the extracellular vesicles, and thus not the recipient of the extracellular vesicles or a product made using the extracellular vesicles.

“Pancreatic islet” or “islet” as provided herein is used according to its common meaning in the art and refers to regions in the pancreas that include hormone-producing cells (e.g. alpha cells, beta cells, delta cells, etc.). Hormones produced in the pancreatic islet include insulin, glucagon, somatostatin, and gastrin, etc. In certain immune disorders (e.g. T1D) cells of the pancreatic islet are destroyed.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents and methods well-known in the art (e.g. flow cytometry, etc.).

A “regulatory T cell”, “suppressor T cell”, or “Treg” is a subpopulation of T cells which modulate the immune system, maintain tolerance to self-antigens, and prevent autoimmune disease. Treg suppress activation of the immune system and typically express markers including CD4, CD25, and Foxp3, and express low or no CD127. Tregs suppresses pro-inflammatory cytokine production and proliferation of effector cells. Some Tregs co-express IFN-g or IL-17. They can proliferate when stimulated with TCR. According to antigen specificity that Tregs recognize, Tregs can be classified into polyclonal Tregs and antigen-specific Tregs.

An “autologous regulatory T cell” or “autologous Treg” refers to a Treg cell from an individual that is typically both donor and recipient of the Treg or a product made using the Treg. For example, autologous polyclonal Treg may be expanded in vitro, and cultured with autologous antigen presenting cells to generate anergic autologous Treg.

The term “antigen presenting cell” or “APC” refers to a heterogeneous group of immune cells that mediate the cellular immune response by processing and presenting antigens for recognition by certain lymphocytes, for example T cells. The antigens are typically complexed with major histocompatibility complexes (MHCs) on the APC surface for display. APCs include dendritic cells, macrophages, and B cells.

“Antigen-loaded” as used herein in reference to an antigen presenting cell refers to an APC that has processed one or more specific antigens. The antigen-loaded cell may present the processed antigens on an MHC molecule (e.g. an MHC Class I or II molecule). For example, a dendritic cell that has processed autoantigens present in samples taken from the islet environment of a T1D patient is an antigen-loaded DC.

Thus, the term “autologous antigen presenting cell” or “autologous APC” as used herein refers to an APC from an individual that is typically both donor and recipient of the APC or a product made using APC. For example, an autologous APC presents autoantigens through contact with islet-derived extracellular vesicles, thereby forming an activated autologous APC. Similarly, an “autologous T regulatory cell” or “autologous Treg cell” is a Treg cell from an individual that is typically both donor and recipient of the Treg cell or a product made from the Treg cell. For example, a Treg cell from a subject may be contacted with an antigen-loaded APC (e.g. antigen loaded DC), and the resultant reprogrammed Treg cell may be administered back to the same subject.

As used herein, “different autologous antigen presenting cells” or “different autologous APCs” refer to a plurality of cells where two or more of distinct types of APCs are included in the plurality of cells. The APCs are from an individual that is typically both donor and recipient of the APCs or a product made using the APCs. For example, the different autologous antigen presenting cells may include a combination of two or more types of antigen presenting cells. The two or more types of antigen presenting cells may include macrophages, B cells or dendritic cells.

“Dendritic cell” or “DC” refer to antigen-presenting cells of the immune system. DCs process antigenic material and present it on the cell surface to T cells. There are two types of DC, conventional dendritic cells (previously called myeloid dendritic cells) which secrete IL-12, IL-6, and TNF, and plasmacytoid dendritic cells (pDC) which can produce high amounts of interferon-α. DCs express certain markers such as CD303, CD141, and CD1c.

“Antigen-specific T regulatory cell”, “antigen-specific Treg” or “aTreg” refer to functional Tregs that control immune responses to one or more specific antigens (e.g. microbial, tumoral, or transplantation antigens) following adequate stimulation (e.g. by contacting the Treg with the cognate antigen and/or immunoregulatory cytokines). aTregs may include T cell receptors (TCRs) by which Tregs become activated to become tolerant or suppressive to specific antigens. The antigen may be self or non-self antigens. In embodiments, aTregs may be generated by contacting Tregs with activated antigen presenting cells.

A “polyclonal regulatory T cell” or “polyclonal Treg” refers to Tregs that are not specific to a particular antigen, have activity toward multiple antigens, and/or have specificity to unknown antigens. Polyclonal Tregs may be immune tolerant to multiple antigens. Polyclonal Tregs may be immunosuppressive to multiple antigens. In embodiments, polyclonal Tregs may be generated by expanding Tregs in vitro.

The term “reprogrammed T regulatory cell” or “reprogrammed Treg” refers to Tregs that are educated by exposure (e.g. in vitro, in situ or not in vivo) to antigens (e.g. self or non-self antigens) either directly (e.g. by way of antigen presenting cells) or indirectly to have immunosuppressive or immune-tolerant properties towards those specific antigens. For example, Tregs cultured with antigen presenting dendritic cells (e.g. tolDC generated by pulsing dendritic cells with islet extracellular vesicles), become reprogrammed Tregs that are specifically immune tolerant to one or more antigens on the dendritic cells. Thus, in embodiments, a reprogrammed Treg cell is contacted with an antigen-loaded tolDC in vitro. For example, Tregs educated by exposure to islet EVs are reprogrammed to be specifically immune tolerant to one or more antigens in the EVs. In embodiments, a Treg is contacted to an islet EV in vitro.

Thus, the term “educate” when used in regards to immune cells, refers to the process of inducing the immune cells to produce a certain type of response to one or more molecules (e.g. antigens, cytokines, etc.). For example, contacting Tregs with tolerogenic dendritic cells (tolDCs) educates the Tregs to be immune tolerant or immunosuppressive in response to the antigens on the tolDCs. In embodiments, Tregs that are educated to become reprogrammed Tregs express higher levels of immunosuppressive cytokines compared to Tregs that have not been educated.

“Monocyte” refers to immune system cells involved in both innate and adaptive immune responses. Monocytes may differentiate into either monocyte-derived macrophages or monocyte-derived dendritic cells. Monocytes may be identified by expression of CD14, which is a co-receptor for toll-like receptor 4 (TLR4) and may mediate lipopolysaccharide (LPS) signaling; and CD16 (Fc gamma receptor IIIa). Monocytes may be identified by three populations, for example, classical (CD14⁺⁺, CD16⁻), intermediate (CD14⁺, CD16⁺), and non-classical (CD14⁺, CD16⁺⁺) monocytes. In embodiments, CD14 monocytes are autologous monocytes. In embodiments, CD14⁺ monocytes are heterologous monocytes. Thus, the term “CD14+ monocyte” refers to a subset of immune cells, including dendritic cells and macrophages, that express the CD14 protein.

The term “chimeric antigen receptor” or “CAR” as provided herein refers to a recombinant protein including an antigen-binding or antigen-recognition domain that targets a certain type of cell (e.g. beta cells) by associating with a protein (e.g. beta cell antigen) on the cell surface. Typically, the antigen-recognition domain of a CAR is derived from a sequence of a portion of a monoclonal antibody (mAb). The antigen-recognition domain is usually attached to a hinge or spacer portion, and the hinge or spacer is attached to a transmembrane domain, which is usually attached to a signaling domain. The CAR may target a single antigen, two antigens, or multiple antigens. Thus, a bispecific CAR refers to a CAR including a first and a second antibody region that interact with a first and second antigen, respectively. In embodiments, the first and second antigens are different antigens. In embodiments, the first and second antigens are different epitopes of the same antigen.

### Methods of Making Reprogrammed Immune Cells

Provided herein, inter alia, are methods for making reprogrammed immune cells. The methods include contacting dendritic cells with antigens (e.g. antigens from the pancreatic islet environment) to have immune-suppressive properties, thereby forming tolerogenic dendritic cells (tolDC). The tolDCs may be used to generate antigen-specific Tregs (also referred to herein as reprogrammed Tregs). In embodiments, the reprogrammed Tregs are specific for antigens in the pancreatic islet environment. Thus, in an aspect is provided a method of generating a reprogrammed T regulatory (Treg) cell, the method including: (a) contacting a dendritic cell (DC) with interleukin-10 (IL-10), thereby producing a tolerogenic dendritic cell (tolDC); and (b) contacting the tolDC with an islet extracellular vesicle (EV), thereby producing an antigen-loaded tolDC; and (c) contacting a Treg cell with the antigen-loaded tolDC, thereby producing the reprogrammed Treg cell.

In embodiments, a Treg is obtained from a subject having T1D. In embodiments after generating a reprogrammed Treg cell from the Treg cell, the reprogrammed Treg cell is administered back into the T1D subject (e.g. a T1D patient). T In embodiments, the DC is obtained from a subject with Type 1 diabetes. In embodiments, the Treg cell and the DC are obtained from the same subject (e.g. a T1D patient). In embodiments, the islet EV is obtained from a donor that is not the subject. In embodiments, the donor does not have Type 1 diabetes. In embodiments, the islet EV includes (e.g. carries cargo) one or more antigens, such as protein antigens. In embodiments, the EV including one or more of an autoantigen, cytokine (e.g. IFN-γ, IL-1, IL-17, IL-6)), lipid or nucleic acid (e.g. miRNA).

In embodiments, the DC of step (a) are further contacted with one or more of Granulocyte-Macrophage Colony Stimulating Factor (GM-CSF), IL-4, interferon-α (IFN-α), ICOS ligand, and CD46. In embodiments, the DC of step (a) are further contacted with fetal bovine serum. In embodiments, the contacting of step (a) occurs for about 1 day. In embodiments, the contacting occurs for about 2 days. In embodiments, the contacting occurs for about 3 days. In embodiments, the contacting occurs for about 4 days. In embodiments, the contacting occurs for about 5 days. In embodiments, the contacting occurs for about 6 days. In embodiments, the contacting occurs for about 7 days. In embodiments, the contacting occurs for about 8 days. In embodiments, the contacting occurs for about 9 days.

In embodiments, the DC of step (a) are further contacted with one or more of GM-CSF, IL-6, IL-1β, TNFα and (Prostaglandin E₂) PGE-2 following the contacting with IL-10. In embodiments, the DC of step (a) are further contacted with GM-CSF, following the contacting with IL-10. In embodiments, the DC of step (a) are further contacted with IL-6. following the contacting with IL-10. In embodiments, the DC of step (a) are further contacted with IL-1β following the contacting with IL-10. In embodiments, the DC of step (a) are further contacted with IL-1 following the contacting with IL-10. In embodiments, the DC of step (a) are further contacted with TNFα following the contacting with IL-10. In embodiments, the DC of step (a) are further contacted PGE-2 following the contacting with IL-10.

In embodiments, the DC of step (a) are contacted with IL-10 in the presence of one or more of GM-CSF, IL-6, IL-1β, TNFα and (Prostaglandin E₂) PGE-2. In embodiments, the DC of step (a) are contacted with IL-10 in the presence of GM-CSF. In embodiments, the DC of step (a) are contacted with IL-10 in the presence of IL-10. In embodiments, the DC of step (a) contacted with IL-10 in the presence of IL-1β. In embodiments, the DC of step (a) contacted with IL-10 in the presence of IL-1. In embodiments, the DC of step (a) are contacted with IL-10 in the presence of TNFα. In embodiments, the DC of step (a) are contacted with IL-10 in the presence of PGE-2.

In embodiments, the islet EV includes one or more of an autoantigen. “Autoantigen” as provided herein is used according to its common meaning in the art. In embodiments, the autoantigen is a protein or group of proteins that is recognized by immune cells of a subject who has an autoimmune disease. In embodiments, the autoantigen is a cause of the autoimmune disease (e.g. TD1). In embodiments the autoantigen is glutamic acid decarboxylase 65 (GAD65) or fragment thereof, islet antigen 2 (IA2) or fragment thereof, non-specific islet cell AAgs (ICA) or fragment thereof, heat shock protein (HSP) or fragment thereof, islet-specific glucose-6-phosphatase catalytic subunit related protein (IGRP) or fragment thereof or imogen-38 or fragment thereof. In embodiments the autoantigen is GAD65 or fragment thereof. In embodiments the autoantigen is IA2 or fragment thereof. In embodiments the autoantigen is ICA or fragment thereof. In embodiments the autoantigen is HSP or fragment thereof. In embodiments the autoantigen is IGRP or fragment thereof. In embodiments the autoantigen is imogen-38 or fragment thereof. In embodiments, the autoantigen is PDX1 or a fragment thereof. In embodiments, the autoantigen is ZnT8 or a fragment thereof. In embodiments, the autoantigen is CHGA0 or a fragment thereof. In embodiments, the autoantigen is IAAP or a fragment thereof.

In embodiments, the Treg cells from the subject (e.g. patient) are expanded prior to contacting with the antigen-loaded tolDC. In embodiments, the Treg cells are expanded from 2 fold to more than 200 fold compared to the original number or concentration of Treg cells in the cell culture. In embodiments, the Treg cells are expanded from 8 fold to more than 200 fold compared to the original number or concentration of Treg cells in the cell culture. In embodiments, the Treg cells are expanded from 16 fold to more than 200 fold compared to the original number or concentration of Treg cells in the cell culture. In embodiments, the Treg cells are expanded from 32 fold to more than 200 fold compared to the original number or concentration of Treg cells in the cell culture. In embodiments, the Treg cells are expanded from 40 fold to more than 200 fold compared to the original number or concentration of Treg cells in the cell culture. In embodiments, the Treg cells are expanded from 48 fold to more than 200 fold compared to the original number or concentration of Treg cells in the cell culture. In embodiments, the Treg cells are expanded from 56 fold to more than 200 fold compared to the original number or concentration of Treg cells in the cell culture. In embodiments, the Treg cells are expanded from 64 fold to more than 200 fold compared to the original number or concentration of Treg cells in the cell culture. In embodiments, the Treg cells are expanded from 72 fold to more than 200 fold compared to the original number or concentration of Treg cells in the cell culture. In embodiments, the Treg cells are expanded from 88 fold to more than 200 fold compared to the original number or concentration of Treg cells in the cell culture. In embodiments, the Treg cells are expanded from 98 fold to more than 200 fold compared to the original number or concentration of Treg cells in the cell culture. In embodiments, the Treg cells are expanded from 104 fold to more than 200 fold compared to the original number or concentration of Treg cells in the cell culture. In embodiments, the Treg cells are expanded from 112 fold to more than 200 fold compared to the original number or concentration of Treg cells in the cell culture. In embodiments, the Treg cells are expanded from 120 fold to more than 200 fold compared to the original number or concentration of Treg cells in the cell culture. In embodiments, the Treg cells are expanded from 128 fold to more than 200 fold compared to the original number or concentration of Treg cells in the cell culture. In embodiments, the Treg cells are expanded from 136 fold to more than 200 fold compared to the original number or concentration of Treg cells in the cell culture. In embodiments, the Treg cells are expanded from 144 fold to more than 200 fold compared to the original number or concentration of Treg cells in the cell culture. In embodiments, the Treg cells are expanded from 152 fold to more than 200 fold compared to the original number or concentration of Treg cells in the cell culture. In embodiments, the Treg cells are expanded from 160 fold to more than 200 fold compared to the original number or concentration of Treg cells in the cell culture. In embodiments, the Treg cells are expanded from 168 fold to more than 200 fold compared to the original number or concentration of Treg cells in the cell culture. In embodiments, the Treg cells are expanded from 176 fold to more than 200 fold compared to the original number or concentration of Treg cells in the cell culture. In embodiments, the Treg cells are expanded from 184 fold to more than 200 fold compared to the original number or concentration of Treg cells in the cell culture. In embodiments, the Treg cells are expanded from 192 fold to more than 200 fold compared to the original number or concentration of Treg cells in the cell culture.

In embodiments, the Treg cells are expanded from 2 fold to 192 fold compared to the original number or concentration of Treg cells in the cell culture. In embodiments, the Treg cells are expanded from 2 fold to 184 fold compared to the original number or concentration of Treg cells in the cell culture. In embodiments, the Treg cells are expanded from 2 fold to 176 fold compared to the original number or concentration of Treg cells in the cell culture. In embodiments, the Treg cells are expanded from 2 fold to 168 fold compared to the original number or concentration of Treg cells in the cell culture. In embodiments, the Treg cells are expanded from 2 fold to 160 fold compared to the original number or concentration of Treg cells in the cell culture. In embodiments, the Treg cells are expanded from 2 fold to 152 fold compared to the original number or concentration of Treg cells in the cell culture. In embodiments, the Treg cells are expanded from 2 fold to 144 fold compared to the original number or concentration of Treg cells in the cell culture. In embodiments, the Treg cells are expanded from 2 fold to 136 fold compared to the original number or concentration of Treg cells in the cell culture. In embodiments, the Treg cells are expanded from 2 fold to 128 fold compared to the original number or concentration of Treg cells in the cell culture. In embodiments, the Treg cells are expanded from 2 fold to 120 fold compared to the original number or concentration of Treg cells in the cell culture. In embodiments, the Treg cells are expanded from 2 fold to 112 fold compared to the original number or concentration of Treg cells in the cell culture. In embodiments, the Treg cells are expanded from 2 fold to 104 fold compared to the original number or concentration of Treg cells in the cell culture. In embodiments, the Treg cells are expanded from 2 fold to 88 fold compared to the original number or concentration of Treg cells in the cell culture. In embodiments, the Treg cells are expanded from 2 fold to 80 fold compared to the original number or concentration of Treg cells in the cell culture. In embodiments, the Treg cells are expanded from 2 fold to 72 fold compared to the original number or concentration of Treg cells in the cell culture. In embodiments, the Treg cells are expanded from 2 fold to 64 fold compared to the original number or concentration of Treg cells in the cell culture. In embodiments, the Treg cells are expanded from 2 fold to 56 fold compared to the original number or concentration of Treg cells in the cell culture. In embodiments, the Treg cells are expanded from 2 fold to 48 fold compared to the original number or concentration of Treg cells in the cell culture. In embodiments, the Treg cells are expanded from 2 fold to 40 fold compared to the original number or concentration of Treg cells in the cell culture. In embodiments, the Treg cells are expanded from 2 fold to 32 fold compared to the original number or concentration of Treg cells in the cell culture. In embodiments, the Treg cells are expanded from 2 fold to 24 fold compared to the original number or concentration of Treg cells in the cell culture. In embodiments, the Treg cells are expanded from 2 fold to 16 fold compared to the original number or concentration of Treg cells in the cell culture. In embodiments, the Treg cells are expanded from 2 fold to 8 fold compared to the original number or concentration of Treg cells in the cell culture. In embodiments, the Treg cells are expanded from 2 fold, 8 fold, 16 fold, 24 fold, 32 fold, 40 fold, 48 fold, 56 fold, 64 fold, 72 fold, 80 fold, 88 fold, 96 fold, 104 fold, 112 fold, 120 fold, 128 fold, 136 fold, 144 fold, 152 fold, 160 fold, 168 fold, 176 fold, 184 fold, 192 fold, or 200 fold compared to the original number or concentration of Treg cells in the cell culture. In embodiments, the Treg cells are expanded 100 fold compared to the original number or concentration of Treg cells in the cell culture.

In embodiments, the Treg cells are expanded from 100 fold to more than 3000 fold compared to the original number or concentration of Treg cells in the cell culture. In embodiments, the Treg cells are expanded from 200 fold to more than 3000 fold compared to the original number or concentration of Treg cells in the cell culture. In embodiments, the Treg cells are expanded from 300 fold to more than 3000 fold compared to the original number or concentration of Treg cells in the cell culture. In embodiments, the Treg cells are expanded from 400 fold to more than 3000 fold compared to the original number or concentration of Treg cells in the cell culture. In embodiments, the Treg cells are expanded from 500 fold to more than 3000 fold compared to the original number or concentration of Treg cells in the cell culture. In embodiments, the Treg cells are expanded from 600 fold to more than 3000 fold compared to the original number or concentration of Treg cells in the cell culture. In embodiments, the Treg cells are expanded from 700 fold to more than 3000 fold compared to the original number or concentration of Treg cells in the cell culture. In embodiments, the Treg cells are expanded from 800 fold to more than 3000 fold compared to the original number or concentration of Treg cells in the cell culture. In embodiments, the Treg cells are expanded from 900 fold to more than 3000 fold compared to the original number or concentration of Treg cells in the cell culture. In embodiments, the Treg cells are expanded from 1000 fold to more than 3000 fold compared to the original number or concentration of Treg cells in the cell culture. In embodiments, the Treg cells are expanded from 1100 fold to more than 3000 fold compared to the original number or concentration of Treg cells in the cell culture. In embodiments, the Treg cells are expanded from 1200 fold to more than 3000 fold compared to the original number or concentration of Treg cells in the cell culture. In embodiments, the Treg cells are expanded from 1300 fold to more than 3000 fold compared to the original number or concentration of Treg cells in the cell culture. In embodiments, the Treg cells are expanded from 1400 fold to more than 3000 fold compared to the original number or concentration of Treg cells in the cell culture. In embodiments, the Treg cells are expanded from 1500 fold to more than 3000 fold compared to the original number or concentration of Treg cells in the cell culture. In embodiments, the Treg cells are expanded from 1600 fold to more than 3000 fold compared to the original number or concentration of Treg cells in the cell culture. In embodiments, the Treg cells are expanded from 1700 fold to more than 3000 fold compared to the original number or concentration of Treg cells in the cell culture. In embodiments, the Treg cells are expanded from 1800 fold to more than 3000 fold compared to the original number or concentration of Treg cells in the cell culture. In embodiments, the Treg cells are expanded from 1900 fold to more than 3000 fold compared to the original number or concentration of Treg cells in the cell culture. In embodiments, the Treg cells are expanded from 2000 fold to more than 3000 fold compared to the original number or concentration of Treg cells in the cell culture. In embodiments, the Treg cells are expanded from 2100 fold to more than 3000 fold compared to the original number or concentration of Treg cells in the cell culture. In embodiments, the Treg cells are expanded from 2200 fold to more than 3000 fold compared to the original number or concentration of Treg cells in the cell culture. In embodiments, the Treg cells are expanded from 2300 fold to more than 3000 fold compared to the original number or concentration of Treg cells in the cell culture. In embodiments, the Treg cells are expanded from 2400 fold to more than 3000 fold compared to the original number or concentration of Treg cells in the cell culture. In embodiments, the Treg cells are expanded from 2500 fold to more than 3000 fold compared to the original number or concentration of Treg cells in the cell culture. In embodiments, the Treg cells are expanded from 2600 fold to more than 3000 fold compared to the original number or concentration of Treg cells in the cell culture. In embodiments, the Treg cells are expanded from 2700 fold to more than 3000 fold compared to the original number or concentration of Treg cells in the cell culture. In embodiments, the Treg cells are expanded from 2800 fold to more than 3000 fold compared to the original number or concentration of Treg cells in the cell culture. In embodiments, the Treg cells are expanded from 2900 fold to more than 3000 fold compared to the original number or concentration of Treg cells in the cell culture.

In embodiments, the Treg cells are expanded from 100 fold to more than 2900 fold compared to the original number or concentration of Treg cells in the cell culture. In embodiments, the Treg cells are expanded from 100 fold to more than 2800 fold compared to the original number or concentration of Treg cells in the cell culture. In embodiments, the Treg cells are expanded from 100 fold to more than 2700 fold compared to the original number or concentration of Treg cells in the cell culture. In embodiments, the Treg cells are expanded from 100 fold to more than 2600 fold compared to the original number or concentration of Treg cells in the cell culture. In embodiments, the Treg cells are expanded from 100 fold to more than 2500 fold compared to the original number or concentration of Treg cells in the cell culture. In embodiments, the Treg cells are expanded from 100 fold to more than 2400 fold compared to the original number or concentration of Treg cells in the cell culture. In embodiments, the Treg cells are expanded from 100 fold to more than 2300 fold compared to the original number or concentration of Treg cells in the cell culture. In embodiments, the Treg cells are expanded from 100 fold to more than 2200 fold compared to the original number or concentration of Treg cells in the cell culture. In embodiments, the Treg cells are expanded from 100 fold to more than 2100 fold compared to the original number or concentration of Treg cells in the cell culture. In embodiments, the Treg cells are expanded from 100 fold to more than 2000 fold compared to the original number or concentration of Treg cells in the cell culture. In embodiments, the Treg cells are expanded from 100 fold to more than 1900 fold compared to the original number or concentration of Treg cells in the cell culture. In embodiments, the Treg cells are expanded from 100 fold to more than 1800 fold compared to the original number or concentration of Treg cells in the cell culture. In embodiments, the Treg cells are expanded from 100 fold to more than 1700 fold compared to the original number or concentration of Treg cells in the cell culture. In embodiments, the Treg cells are expanded from 100 fold to more than 1600 fold compared to the original number or concentration of Treg cells in the cell culture. In embodiments, the Treg cells are expanded from 100 fold to more than 1500 fold compared to the original number or concentration of Treg cells in the cell culture. In embodiments, the Treg cells are expanded from 100 fold to more than 1400 fold compared to the original number or concentration of Treg cells in the cell culture. In embodiments, the Treg cells are expanded from 100 fold to more than 1300 fold compared to the original number or concentration of Treg cells in the cell culture. In embodiments, the Treg cells are expanded from 100 fold to more than 1200 fold compared to the original number or concentration of Treg cells in the cell culture. In embodiments, the Treg cells are expanded from 100 fold to more than 1100 fold compared to the original number or concentration of Treg cells in the cell culture. In embodiments, the Treg cells are expanded from 100 fold to more than 1000 fold compared to the original number or concentration of Treg cells in the cell culture. In embodiments, the Treg cells are expanded from 100 fold to more than 900 fold compared to the original number or concentration of Treg cells in the cell culture. In embodiments, the Treg cells are expanded from 100 fold to more than 800 fold compared to the original number or concentration of Treg cells in the cell culture. In embodiments, the Treg cells are expanded from 100 fold to more than 700 fold compared to the original number or concentration of Treg cells in the cell culture. In embodiments, the Treg cells are expanded from 100 fold to more than 600 fold compared to the original number or concentration of Treg cells in the cell culture. In embodiments, the Treg cells are expanded from 100 fold to more than 500 fold compared to the original number or concentration of Treg cells in the cell culture. In embodiments, the Treg cells are expanded from 100 fold to more than 400 fold compared to the original number or concentration of Treg cells in the cell culture. In embodiments, the Treg cells are expanded from 100 fold to more than 300 fold compared to the original number or concentration of Treg cells in the cell culture. In embodiments, the Treg cells are expanded from 100 fold to more than 200 fold compared to the original number or concentration of Treg cells in the cell culture. In embodiments, the Treg cells are expanded from 100 fold, 200 fold, 300 fold, 400 fold, 500 fold, 600 fold, 700 fold, 800 fold, 900 fold, 1000 fold, 1100 fold, 1200 fold, 1300 fold, 1400 fold, 1500 fold, 1600 fold, 1700 fold, 1800 fold, 1900 fold, 2000 fold, 2100 fold, 2200 fold, 2300 fold, 2400 fold, 2500 fold, 2600 fold, 2700 fold, 2800 fold, 2900 fold, or 3000 fold, compared to the original number or concentration of Treg cells in the cell culture. In embodiments, the Treg cells are expanded 600 fold compared to the original number or concentration of Treg cells in the cell culture.

In embodiments, the Treg cell from the donor are expanded from 2 days to 28 days. In embodiments, the Treg cell from the donor are expanded from 4 days to 28 days. In embodiments, the Treg cell from the donor are expanded from 6 days to 28 days. In embodiments, the Treg cell from the donor are expanded from 8 days to 28 days. In embodiments, the Treg cell from the donor are expanded from 10 days to 28 days. In embodiments, the Treg cell from the donor are expanded from 12 days to 28 days. In embodiments, the Treg cell from the donor are expanded from 14 days to 28 days. In embodiments, the Treg cell from the donor are expanded from 16 days to 28 days. In embodiments, the Treg cell from the donor are expanded from 18 days to 28 days. In embodiments, the Treg cell from the donor are expanded from 20 days to 28 days. In embodiments, the Treg cell from the donor are expanded from 22 days to 28 days. In embodiments, the Treg cell from the donor are expanded from 24 days to 28 days. In embodiments, the Treg cell from the donor are expanded from 26 days to 28 days.

In embodiments, the Treg cell from the donor are expanded from 2 days to 26 days. In embodiments, the Treg cell from the donor are expanded from 2 days to 24 days. In embodiments, the Treg cell from the donor are expanded from 2 days to 22 days. In embodiments, the Treg cell from the donor are expanded from 2 days to 20 days. In embodiments, the Treg cell from the donor are expanded from 2 days to 18 days. In embodiments, the Treg cell from the donor are expanded from 2 days to 16 days. In embodiments, the Treg cell from the donor are expanded from 2 days to 14 days. In embodiments, the Treg cell from the donor are expanded from 2 days to 12 days. In embodiments, the Treg cell from the donor are expanded from 2 days to 10 days. In embodiments, the Treg cell from the donor are expanded from 2 days to 8 days. In embodiments, the Treg cell from the donor are expanded from 2 days to 6 days. In embodiments, the Treg cell from the donor are expanded from 2 days to 4 days. In embodiments, the Treg cell from the donor are expanded for 2 days, 4 days, 6 days, 8 days, 10 days, 12 days, 14 days, 16 days, 18 days, 20 days, 22 days, 24 days, 26 days, or 28 days. In embodiments, the Treg cell from the donor are expanded for 21 days.

In embodiments, the Tregs are contacted with the antigen-loaded tolDC at a ratio of 10:1 (Treg:DC). In embodiments, the Tregs are contacted with the antigen-loaded tolDC at a ratio of 9:1 (Treg:DC). In embodiments, the Tregs are contacted with the antigen-loaded tolDC at a ratio of 8:1 (Treg:DC). In embodiments, the Tregs are contacted with the antigen-loaded tolDC at a ratio of 7:1 (Treg:DC). In embodiments, the Tregs are contacted with the antigen-loaded tolDC at a ratio of 6:1 (Treg:DC). In embodiments, the Tregs are contacted with the antigen-loaded tolDC at a ratio of 5:1 (Treg:DC). In embodiments, the Tregs are contacted with the antigen-loaded tolDC at a ratio of 4:1 (Treg:DC). In embodiments, the Tregs are contacted with the antigen-loaded tolDC at a ratio of 3:1 (Treg:DC). In embodiments, the Tregs are contacted with the antigen-loaded tolDC at a ratio of 2:1 (Treg:DC). In embodiments, the Tregs are contacted with the antigen-loaded tolDC at a ratio of 1:1 (Treg:DC). In embodiments, the Tregs are contacted with the antigen-loaded tolDC at a ratio of 0.5:1 (Treg:DC). In embodiments, the Tregs are contacted with the antigen-loaded tolDC at a ratio of 0.25:1 (Treg:DC).

In embodiments, step (a) further includes expanding the tolDC. In embodiments, step (b) further includes expanding the antigen-loaded tolDC. In embodiments, step (c) further includes expanding the reprogrammed Treg cell.

In embodiments, the reprogrammed Treg is expanded from 2 days to 28 days. In embodiments, the reprogrammed Treg is expanded from 4 days to 28 days. In embodiments, the reprogrammed Treg is expanded from 6 days to 28 days. In embodiments, the reprogrammed Treg is expanded from 8 days to 28 days. In embodiments, the reprogrammed Treg is expanded from 10 days to 28 days. In embodiments, the reprogrammed Treg is expanded from 12 days to 28 days. In embodiments, the reprogrammed Treg is expanded from 14 days to 28 days. In embodiments, the reprogrammed Treg is expanded from 16 days to 28 days. In embodiments, the reprogrammed Treg is expanded from 18 days to 28 days. In embodiments, the reprogrammed Treg is expanded from 20 days to 28 days. In embodiments, the reprogrammed Treg is expanded from 22 days to 28 days. In embodiments, the reprogrammed Treg is expanded from 24 days to 28 days. In embodiments, the reprogrammed Treg is expanded from 26 days to 28 days.

In embodiments, the reprogrammed Treg is expanded from 2 days to 26 days. In embodiments, the reprogrammed Treg is expanded from 2 days to 24 days. In embodiments, the reprogrammed Treg is expanded from 2 days to 22 days. In embodiments, the reprogrammed Treg is expanded from 2 days to 20 days. In embodiments, the reprogrammed Treg is expanded from 2 days to 18 days. In embodiments, the reprogrammed Treg is expanded from 2 days to 16 days. In embodiments, the reprogrammed Treg is expanded from 2 days to 14 days. In embodiments, the reprogrammed Treg is expanded from 2 days to 12 days. In embodiments, the reprogrammed Treg is expanded from 2 days to 10 days. In embodiments, the reprogrammed Treg is expanded from 2 days to 8 days. In embodiments, the reprogrammed Treg is expanded from 2 days to 6 days. In embodiments, the reprogrammed Treg is expanded from 2 days to 4 days. In embodiments, the reprogrammed Treg is expanded for 2 days, 4 days, 6 days, 8 days, 10 days, 12 days, 14 days, 16 days, 18 days, 20 days, 22 days, 24 days, 26 days, or 28 days. In embodiments, the reprogrammed Treg is expanded for 21 days.

In embodiments, the reprogrammed Treg cell is expanded in the presence of IL-2 and/or IL-15. In embodiments, the reprogrammed Treg cell is expanded in the presence of IL-2. In embodiments, the reprogrammed Treg cell is expanded in the presence of IL-15.

In embodiments, the reprogrammed Treg cell expresses higher levels of an immunosuppressive cytokine compared to a polyclonal Treg cell. In embodiments, the reprogrammed Treg cell expresses at least 5% more, 10% more, 20% more, 30% more, 40% more, 50% more, 60% more, 70% more, 80% more, 90% more, 100% more, 120% more, 140% more, 160% more, 180% more, or 200% more of an immunosuppressive cytokine compared to a polyclonal Treg cell. Expression of a cytokine can be detected using conventional techniques for detecting protein (e.g., ELISA, Western blotting, flow cytometry, immunofluorescence, immunohistochemistry, etc.). In embodiments, the immunosuppressive cytokine is IL-10 and/or tumor growth factor-β(TGF-β). In embodiments, the immunosuppressive cytokine is IL-10. In embodiments, the immunosuppressive cytokine is TGF-β.

In embodiments, the reprogrammed Treg cell expresses lower levels of a proinflammatory cytokine compared to a polyclonal Treg. In embodiments, the reprogrammed Treg cell expresses at least 5% less, 10% less, 20% less, 30% less, 40% less, 50% less, 60% less, 70% less, 80% less, 90% less, 95% less, 98% less or 99% less of an immunosuppressive cytokine compared to a polyclonal Treg cell. In embodiments, the proinflammatory cytokine is interferon-γ (IFN-γ), tumor necrosis factor-α (TNF-α), interleukin-4 (IL-4), and/or interleukin-6 (IL-6). In embodiments, the proinflammatory cytokine is IFN-γ. In embodiments, the proinflammatory cytokine is TNF-α. In embodiments, the proinflammatory cytokine is IL-4. In embodiments, the proinflammatory cytokine is IL-6.

In embodiments, the reprogrammed Treg cell has immune suppressive properties. In embodiments, the immune suppressive properties include inhibiting proliferation of effector T cells (e.g. cytotoxic T cells, CTLs, T-killer cells, killer T cells). Thus, in embodiments, the reprogrammed Treg cell increases suppression of CD4+ and CD8+ T cell proliferation compared to a polyclonal Treg cell. In embodiments, the reprogrammed Treg cell suppresses CD4+ and CD8+ T cell proliferation at least 2.5% more, 5% more, 10% more, 20% more, 30% more, 40% more, 50% more, 60% more, 70% more, 80% more, 90% more, 95% more, or 98% more compared to a polyclonal Treg cell.

In embodiments, the reprogrammed Treg cell is contacted with a nucleic acid encoding an antibody region, chimeric antibody receptor (CAR) or portions thereof provided herein including embodiments thereof. In embodiments, the nucleic acid is RNA or DNA. In embodiments, the nucleic acid includes an expression vector. In embodiments, the expression vector is a viral vector or a plasmid. In embodiments, the vector is a viral vector. In embodiments, the vector is a plasmid.

In an aspect is provided a method of generating tolerogenic dendritic cells (tolDCs) including contacting islet extracellular vesicles (islet-EVs) with CD14+ monocytes (e.g. dendritic cells), wherein the contacting results in tolDCs. In embodiments, CD14+ monocytes are obtained from a subject with Type 1 diabetes. In embodiments, the contacting is performed in the presence of interleukin-10 (IL-10). In an aspect is provided a method of generating reprogrammed T-regulatory cells (Tregs), the method including contacting isolated polyclonal Tregs with the tolerogenic dendritic cell provided herein, wherein the contacting results in Tregs expressing CD4+, CD25+ and FoxP3. In embodiments, the reprogrammed Tregs secrete higher levels of an immunosuppressive cytokine compared to polyclonal Tregs. In embodiments, the immunosuppressive cytokine is IL-10 or TGF-beta. In embodiments, the immunosuppressive cytokine is IL-10. In embodiments, the immunosuppressive cytokine is TGF-beta.

In embodiments, EVs from cultured islets of a healthy donor or a T1D patient include a plurality of potential islet-specific autoantigens that can be utilized to generate reprogrammed Tregs. In embodiments, EVs from cultured islets of a healthy donor comprise a plurality of potential islet-specific autoantigens that can be utilized to generate reprogrammed Tregs. In embodiments, the reprogramming with a plurality of autoantigens compared to aTregs reprogrammed with a single autoantigen result in more potent aTregs.

In embodiments, the reprogrammed Tregs secrete lower levels of a proinflammatory cytokine. In embodiments, the proinflammatory cytokine is interferon-gamma (IFN-gamma), tumor necrosis factor alpha (TNF-alpha), interleukin-4 (IL-4), or interleukin-6 (IL-6). In embodiments, the proinflammatory cytokine is IFN-gamma. In embodiments, the proinflammatory cytokine is TNF-alpha. In embodiments, the proinflammatory cytokine is IL-4. In embodiments, the proinflammatory cytokine is IL-6.

In embodiments, the reprogrammed Tregs increase suppression of CD4+ and CD8+ T cell proliferation compared to polyclonal Tregs. In embodiments, the reprogrammed Tregs increase suppression of CD4+ T cell proliferation compared to polyclonal Tregs. In embodiments, the reprogrammed Tregs increase suppression of CD8+ T cell proliferation compared to polyclonal Tregs.

In embodiments, the reprogrammed Tregs specifically target the pancreatic islet microenvironment. In embodiments, the reprogrammed Tregs target beta cells.

In an aspect is provided a method of treating Type 1 diabetes in a subject, the method including administering the reprogrammed Treg provide herein including embodiments thereof to the subject.

### Compositions Including Reprogrammed Immune Cells

Provided herein are reprogrammed immune cells (e.g. reprogrammed Treg cells) that are contemplated to modulate the immune response in a subject who has an autoimmune disease (e.g. Type 1 diabetes). In embodiments, the reprogrammed immune cells express higher levels of anti-inflammatory cytokines and lower levels of pro-inflammatory cytokines. Thus, in an aspect is provided a reprogrammed Treg cell made by a method provided herein including embodiments thereof.

In embodiments, the reprogrammed Treg cell expresses higher levels of an immunosuppressive cytokine compared to a polyclonal Treg cell. In embodiments, the reprogrammed Treg cell expresses at least 5% more, 10% more, 20% more, 30% more, 40% more, 50% more, 60% more, 70% more, 80% more, 90% more, 100% more, 120% more, 140% more, 160% more, 180% more, or 200% more of an immunosuppressive cytokine compared to a polyclonal Treg cell. In embodiments, the immunosuppressive cytokine is IL-10 and/or tumor growth factor-β (TGF-β). In embodiments, the immunosuppressive cytokine is IL-10. In embodiments, the immunosuppressive cytokine is TGF-β.

In embodiments, the reprogrammed Treg cell expresses lower levels of a proinflammatory cytokine compared to a polyclonal Treg. In embodiments, the reprogrammed Treg cell expresses at least 5% less, 10% less, 20% less, 30% less, 40% less, 50% less, 60% less, 70% less, 80% less, 90% less, 95% less, 98% less or 99% less of an immunosuppressive cytokine compared to a polyclonal Treg cell. In embodiments, the proinflammatory cytokine is interferon-γ (IFN-γ), tumor necrosis factor-α (TNF-α), interleukin-4 (IL-4), and/or interleukin-6 (IL-6). In embodiments, the proinflammatory cytokine is IFN-γ. In embodiments, the proinflammatory cytokine is TNF-α. In embodiments, the proinflammatory cytokine is IL-4. In embodiments, the proinflammatory cytokine is IL-6.

The reprogrammed Treg cell provided herein including embodiments thereof may include one or more protein binding domains that bind to a protein on the surface of a target cell. For example, the reprogrammed Treg cell may express an antibody domain that binds a protein expressed on the surface of a beta cell in the pancreatic islet environment, thereby trafficking the reprogrammed Treg cells to the pancreas. Thus, in embodiments, the reprogrammed Treg cell targets a protein expressed on the surface of a pancreatic beta cell. In embodiments, the protein is Sodium/potassium-transporting ATPase subunit gamma (FXYD2), NTPDase3, Glucagon-like peptide-1 receptor (GLP-1R) or GPR44 binding domain. In embodiments, the protein is FXYD2. In embodiments, the protein is NTPDase3. In embodiments, the protein is GLP-1R. In embodiments, the protein is GPR44.

In embodiments, the reprogrammed Treg cell provided herein including embodiments thereof, includes a chimeric antigen receptor (CAR). For example, the CAR may include an antibody region that binds to a protein expressed on the surface of a target cell. In embodiments, the CAR includes an (i) antibody region including a FXYD2, NTPDase3, GLP-1R or GPR44 binding domain; and (ii) a transmembrane binding domain. In embodiments, the CAR includes an antibody region including a FXYD2 binding domain. In embodiments, the CAR includes an antibody region including a NTPDase3 binding domain. In embodiments, the CAR includes an antibody region including a GLP-1R binding domain. In embodiments, the CAR includes an antibody region including a GPR44 binding domain.

An “antibody region” as provided herein refers to a monovalent or multivalent protein moiety that forms part of the recombinant protein (e.g., CAR, bispecific antibody) provided herein including embodiments thereof. A person of ordinary skill in the art will therefore immediately recognize that the antibody region is a protein moiety capable of binding an antigen (epitope). Thus, the antibody region provided herein may include a domain of an antibody (e.g., a light chain variable (VL) domain, a heavy chain variable (VH) domain) or a fragment of an antibody (e.g., Fab). In embodiments, the antibody region is a protein conjugate. A “protein conjugate” as provided herein refers to a construct consisting of more than one polypeptide, wherein the polypeptides are bound together covalently or non-covalently. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv.

As used herein, “GPR44 binding domain” refers to the portion of an antibody, or a functional fragment thereof that recognizes and binds a GPR44 epitope. Typically, the antibody portion that binds the GPR44 epitope comprises the complementarity-determining regions (CDRs) within the variable chains of the antibody.

Similarly, as used herein, “CD4 binding domain” refers to the portion of an antibody, or a functional fragment thereof that recognizes and binds a CD4 epitope. Typically, the antibody portion that binds the CD4 epitope comprises the complementarity-determining regions (CDRs) within the variable chains of the antibody.

The ability of an antibody region to bind a specific epitope (e.g., FXYD2, NTPDase3, GLP-1R, GPR44) can be described by the equilibrium dissociation constant (K_{D}). The equilibrium dissociation constant (K_{D}) as defined herein is the ratio of the dissociation rate (K-off) and the association rate (K-on) of an antibody region to a protein. It is described by the following formula: K_{D}=K-off/K-on.

In embodiments, the antibody region binds a target protein (e.g. FXYD2, NTPDase3, GLP-1R, GPR44) with an equilibrium dissociation constant (K_{D}) from 0.01 nM to 10 nM. In embodiments, the antibody region binds a target protein with a K_{D} from 0.1 nM to 10 nM. In embodiments, the antibody region binds a target protein with a K_{D} from 0.2 nM to 10 nM. In embodiments, the antibody region binds a target protein with a K_{D} from 0.3 nM to 10 nM. In embodiments, the antibody region binds a target protein with a K_{D} from 0.4 nM to 10 nM. In embodiments, the antibody region binds a target protein with a K_{D} from 0.5 nM to 10 nM. In embodiments, the antibody region binds a target protein with a K_{D} from 0.6 nM to 10 nM. In embodiments, the antibody region binds a target protein with a K_{D} from 0.7 nM to 10 nM. In embodiments, the antibody region binds a target protein with a K_{D} from 0.8 nM to 10 nM. In embodiments, the antibody region binds a target protein with a K_{D} from 0.9 nM to 1 nM. In embodiments, the antibody region binds a target protein with a K_{D} from 1 nM to 10 nM. In embodiments, the antibody region binds a target protein with an equilibrium dissociation constant (K_{D}) from 1.1 nM to 10 nM. In embodiments, the antibody region binds a target protein with a K_{D} from 1.2 nM to 10 nM. In embodiments, the antibody region binds a target protein with a K_{D} from 1.3 nM to 10 nM. In embodiments, the antibody region binds a target protein with a K_{D} from 1.4 nM to 10 nM. In embodiments, the antibody region binds a target protein with a K_{D} from 1.5 nM to 10 nM. In embodiments, the antibody region binds a target protein with a K_{D} from 1.6 nM to 10 nM. In embodiments, the antibody region binds a target protein with a K_{D} from 1.7 nM to 10 nM. In embodiments, the antibody region binds a target protein with a K_{D} from 1.8 nM to 10 nM. In embodiments, the antibody region binds a target protein with a K_{D} from 1.9 nM to 10 nM. In embodiments, the antibody region binds a target protein with a K_{D} from 2 nM to 10 nM. In embodiments, the antibody region binds a target protein with an equilibrium dissociation constant (K_{D}) from 2.1 nM to 10 nM. In embodiments, the antibody region binds a target protein with a K_{D} from 2.2 nM to 10 nM. In embodiments, the antibody region binds a target protein with a K_{D} from 2.3 nM to 10 nM. In embodiments, the antibody region binds a target protein with a K_{D} from 2.4 nM to 10 nM. In embodiments, the antibody region binds a target protein with a K_{D} from 2.5 nM to 10 nM. In embodiments, the antibody region binds a target protein with a K_{D} from 2.6 nM to 10 nM. In embodiments, the antibody region binds a target protein with a K_{D} from 2.7 nM to 10 nM. In embodiments, the antibody region binds a target protein with a K_{D} from 2.8 nM to 10 nM. In embodiments, the antibody region binds a target protein with a K_{D} from 2.9 nM to 10 nM. In embodiments, the antibody region binds a target protein with a K_{D} from 3 nM to 10 nM. In embodiments, the antibody region binds a target protein with an equilibrium dissociation constant (K_{D}) from 4.1 nM to 10 nM. In embodiments, the antibody region binds a target protein with a K_{D} from 4.2 nM to 10 nM. In embodiments, the antibody region binds a target protein with a K_{D} from 4.3 nM to 10 nM. In embodiments, the antibody region binds a target protein with a K_{D} from 4.4 nM to 10 nM. In embodiments, the antibody region binds a target protein with a K_{D} from 4.5 nM to 10 nM. In embodiments, the antibody region binds a target protein with a K_{D} from 4.6 nM to 10 nM. In embodiments, the antibody region binds a target protein with a K_{D} from 4.7 nM to 10 nM. In embodiments, the antibody region binds a target protein with a K_{D} from 4.8 nM to 10 nM. In embodiments, the antibody region binds a target protein with a K_{D} from 4.9 nM to 10 nM. In embodiments, the antibody region binds a target protein with a K_{D} from 5 nM to 10 nM. In embodiments, the antibody region binds a target protein with an equilibrium dissociation constant (K_{D}) from 5.1 nM to 10 nM. In embodiments, the antibody region binds a target protein with a K_{D} from 5.2 nM to 10 nM. In embodiments, the antibody region binds a target protein with a K_{D} from 5.3 nM to 10 nM. In embodiments, the antibody region binds a target protein with a K_{D} from 5.4 nM to 10 nM. In embodiments, the antibody region binds a target protein with a K_{D} from 5.5 nM to 10 nM. In embodiments, the antibody region binds a target protein with a K_{D} from 5.6 nM to 10 nM. In embodiments, the antibody region binds a target protein with a K_{D} from 5.7 nM to 10 nM. In embodiments, the antibody region binds a target protein with a K_{D} from 5.8 nM to 10 nM. In embodiments, the antibody region binds a target protein with a K_{D} from 5.9 nM to 10 nM. In embodiments, the antibody region binds a target protein with a K_{D} from 6 nM to 10 nM. In embodiments, the antibody region binds a target protein with an equilibrium dissociation constant (K_{D}) from 6.1 nM to 10 nM. In embodiments, the antibody region binds a target protein with a K_{D} from 6.2 nM to 10 nM. In embodiments, the antibody region binds a target protein with a K_{D} from 6.3 nM to 10 nM. In embodiments, the antibody region binds a target protein with a K_{D} from 6.4 nM to 10 nM. In embodiments, the antibody region binds a target protein with a K_{D} from 6.5 nM to 10 nM. In embodiments, the antibody region binds a target protein with a K_{D} from 6.6 nM to 10 nM. In embodiments, the antibody region binds a target protein with a K_{D} from 6.7 nM to 10 nM. In embodiments, the antibody region binds a target protein with a K_{D} from 6.8 nM to 10 nM. In embodiments, the antibody region binds a target protein with a K_{D} from 6.9 nM to 10 nM. In embodiments, the antibody region binds a target protein with a K_{D} from 7 nM to 10 nM. In embodiments, the antibody region binds a target protein with an equilibrium dissociation constant (K_{D}) from 7.1 nM to 10 nM. In embodiments, the antibody region binds a target protein with a K_{D} from 7.2 nM to 10 nM. In embodiments, the antibody region binds a target protein with a K_{D} from 7.3 nM to 10 nM. In embodiments, the antibody region binds a target protein with a K_{D} from 7.4 nM to 10 nM. In embodiments, the antibody region binds a target protein with a K_{D} from 7.5 nM to 10 nM. In embodiments, the antibody region binds a target protein with a K_{D} from 7.6 nM to 10 nM. In embodiments, the antibody region binds a target protein with a K_{D} from 7.7 nM to 10 nM. In embodiments, the antibody region binds a target protein with a K_{D} from 7.8 nM to 10 nM. In embodiments, the antibody region binds a target protein with a K_{D} from 7.9 nM to 10 nM. In embodiments, the antibody region binds a target protein with a K_{D} from 8 nM to 10 nM. In embodiments, the antibody region binds a target protein with an equilibrium dissociation constant (K_{D}) from 8.1 nM to 10 nM. In embodiments, the antibody region binds a target protein with a K_{D} from 8.2 nM to 10 nM. In embodiments, the antibody region binds a target protein with a K_{D} from 8.3 nM to 10 nM. In embodiments, the antibody region binds a target protein with a K_{D} from 8.4 nM to 10 nM. In embodiments, the antibody region binds a target protein with a K_{D} from 8.5 nM to 10 nM. In embodiments, the antibody region binds a target protein with a K_{D} from 8.6 nM to 10 nM. In embodiments, the antibody region binds a target protein with a K_{D} from 8.7 nM to 10 nM. In embodiments, the antibody region binds a target protein with a K_{D} from 8.8 nM to 10 nM. In embodiments, the antibody region binds a target protein with a K_{D} from 8.9 nM to 10 nM. In embodiments, the antibody region binds a target protein with a K_{D} from 9 nM to 10 nM. In embodiments, the antibody region binds a target protein with an equilibrium dissociation constant (K_{D}) from 9.1 nM to 10 nM. In embodiments, the antibody region binds a target protein with a K_{D} from 9.2 nM to 10 nM. In embodiments, the antibody region binds a target protein with a K_{D} from 9.3 nM to 10 nM. In embodiments, the antibody region binds a target protein with a K_{D} from 9.4 nM to 10 nM. In embodiments, the antibody region binds a target protein with a K_{D} from 9.5 nM to 10 nM. In embodiments, the antibody region binds a target protein with a K_{D} from 9.6 nM to 10 nM. In embodiments, the antibody region binds a target protein with a K_{D} from 9.7 nM to 10 nM. In embodiments, the antibody region binds a target protein with a K_{D} from 9.8 nM to 10 nM. In embodiments, the antibody region binds a target protein with a K_{D} from 9.9 nM to 10 nM.

In embodiments, the antibody region binds a target protein with a K_{D} from 0.01 nM to 9.9 nM. In embodiments, the antibody region binds a target protein with a K_{D} from 0.01 nM to 9.8 nM. In embodiments, the antibody region binds a target protein with a K_{D} from 0.01 nM to 9.7 nM. In embodiments, the antibody region binds a target protein with a K_{D} from 0.01 nM to 9.6 nM. In embodiments, the antibody region binds a target protein with a K_{D} from 0.01 nM to 9.5 nM. In embodiments, the antibody region binds a target protein with a K_{D} from 0.01 nM to 9.4 nM. In embodiments, the antibody region binds a target protein with a K_{D} from 0.01 nM to 9.3 nM. In embodiments, the antibody region binds a target protein with a K_{D} from 0.01 nM to 9.2 nM. In embodiments, the antibody region binds a target protein with a K_{D} from 0.01 nM to 9.1 nM. In embodiments, the antibody region binds a target protein with a K_{D} from 0.01 nM to 9 nM. In embodiments, the antibody region binds a target protein with a K_{D} from 0.01 nM to 8.9 nM. In embodiments, the antibody region binds a target protein with a K_{D} from 0.01 nM to 8.8 nM. In embodiments, the antibody region binds a target protein with a K_{D} from 0.01 nM to 8.7 nM. In embodiments, the antibody region binds a target protein with a K_{D} from 0.01 nM to 8.6 nM. In embodiments, the antibody region binds a target protein with a K_{D} from 0.01 nM to 8.5 nM. In embodiments, the antibody region binds a target protein with a K_{D} from 0.01 nM to 8.4 nM. In embodiments, the antibody region binds a target protein with a K_{D} from 0.01 nM to 8.3 nM. In embodiments, the antibody region binds a target protein with a K_{D} from 0.01 nM to 8.2 nM. In embodiments, the antibody region binds a target protein with a K_{D} from 0.01 nM to 8.1 nM. In embodiments, the antibody region binds a target protein with a K_{D} from 0.01 nM to 8 nM. In embodiments, the antibody region binds a target protein with a K_{D} from 0.01 nM to 7.9 nM. In embodiments, the antibody region binds a target protein with a K_{D} from 0.01 nM to 7.8 nM. In embodiments, the antibody region binds a target protein with a K_{D} from 0.01 nM to 7.7 nM. In embodiments, the antibody region binds a target protein with a K_{D} from 0.01 nM to 7.6 nM. In embodiments, the antibody region binds a target protein with a K_{D} from 0.01 nM to 7.5 nM. In embodiments, the antibody region binds a target protein with a K_{D} from 0.01 nM to 7.4 nM. In embodiments, the antibody region binds a target protein with a K_{D} from 0.01 nM to 7.3 nM. In embodiments, the antibody region binds a target protein with a K_{D} from 0.01 nM to 7.2 nM. In embodiments, the antibody region binds a target protein with a K_{D} from 0.01 nM to 7.1 nM. In embodiments, the antibody region binds a target protein with a K_{D} from 0.01 nM to 7 nM. In embodiments, the antibody region binds a target protein with a K_{D} from 0.01 nM to 6.9 nM. In embodiments, the antibody region binds a target protein with a K_{D} from 0.01 nM to 6.8 nM. In embodiments, the antibody region binds a target protein with a K_{D} from 0.01 nM to 6.7 nM. In embodiments, the antibody region binds a target protein with a K_{D} from 0.01 nM to 6.6 nM. In embodiments, the antibody region binds a target protein with a K_{D} from 0.01 nM to 6.5 nM. In embodiments, the antibody region binds a target protein with a K_{D} from 0.01 nM to 6.4 nM. In embodiments, the antibody region binds a target protein with a K_{D} from 0.01 nM to 6.3 nM. In embodiments, the antibody region binds a target protein with a K_{D} from 0.01 nM to 6.2 nM. In embodiments, the antibody region binds a target protein with a K_{D} from 0.01 nM to 6.1 nM. In embodiments, the antibody region binds a target protein with a K_{D} from 0.01 nM to 6 nM. In embodiments, the antibody region binds a target protein with a K_{D} from 0.01 nM to 5.9 nM. In embodiments, the antibody region binds a target protein with a K_{D} from 0.01 nM to 5.8 nM. In embodiments, the antibody region binds a target protein with a K_{D} from 0.01 nM to 5.7 nM. In embodiments, the antibody region binds a target protein with a K_{D} from 0.01 nM to 5.6 nM. In embodiments, the antibody region binds a target protein with a K_{D} from 0.01 nM to 5.5 nM. In embodiments, the antibody region binds a target protein with a K_{D} from 0.01 nM to 5.4 nM. In embodiments, the antibody region binds a target protein with a K_{D} from 0.01 nM to 5.3 nM. In embodiments, the antibody region binds a target protein with a K_{D} from 0.01 nM to 5.2 nM. In embodiments, the antibody region binds a target protein with a K_{D} from 0.01 nM to 5.1 nM. In embodiments, the antibody region binds a target protein with a K_{D} from 0.01 nM to 5 nM. In embodiments, the antibody region binds a target protein with a K_{D} from 0.01 nM to 4.9 nM. In embodiments, the antibody region binds a target protein with a K_{D} from 0.01 nM to 4.8 nM. In embodiments, the antibody region binds a target protein with a K_{D} from 0.01 nM to 4.7 nM. In embodiments, the antibody region binds a target protein with a K_{D} from 0.01 nM to 4.6 nM. In embodiments, the antibody region binds a target protein with a K_{D} from 0.01 nM to 4.5 nM. In embodiments, the antibody region binds a target protein with a K_{D} from 0.01 nM to 4.4 nM. In embodiments, the antibody region binds a target protein with a K_{D} from 0.01 nM to 4.3 nM. In embodiments, the antibody region binds a target protein with a K_{D} from 0.01 nM to 4.2 nM. In embodiments, the antibody region binds a target protein with a K_{D} from 0.01 nM to 4.1 nM. In embodiments, the antibody region binds a target protein with a K_{D} from 0.01 nM to 4 nM. In embodiments, the antibody region binds a target protein with a K_{D} from 0.01 nM to 3.9 nM. In embodiments, the antibody region binds a target protein with a K_{D} from 0.01 nM to 3.8 nM. In embodiments, the antibody region binds a target protein with a K_{D} from 0.01 nM to 3.7 nM. In embodiments, the antibody region binds a target protein with a K_{D} from 0.01 nM to 3.6 nM. In embodiments, the antibody region binds a target protein with a K_{D} from 0.01 nM to 3.5 nM. In embodiments, the antibody region binds a target protein with a K_{D} from 0.01 nM to 3.4 nM. In embodiments, the antibody region binds a target protein with a K_{D} from 0.01 nM to 3.3 nM. In embodiments, the antibody region binds a target protein with a K_{D} from 0.01 nM to 3.2 nM. In embodiments, the antibody region binds a target protein with a K_{D} from 0.01 nM to 3.1 nM. In embodiments, the antibody region binds a target protein with a K_{D} from 0.01 nM to 3 nM. In embodiments, the antibody region binds a target protein with a K_{D} from 0.01 nM to 2.9 nM. In embodiments, the antibody region binds a target protein with a K_{D} from 0.01 nM to 2.8 nM. In embodiments, the antibody region binds a target protein with a K_{D} from 0.01 nM to 2.7 nM. In embodiments, the antibody region binds a target protein with a K_{D} from 0.01 nM to 2.6 nM. In embodiments, the antibody region binds a target protein with a K_{D} from 0.01 nM to 2.5 nM. In embodiments, the antibody region binds a target protein with a K_{D} from 0.01 nM to 2.4 nM. In embodiments, the antibody region binds a target protein with a K_{D} from 0.01 nM to 2.3 nM. In embodiments, the antibody region binds a target protein with a K_{D} from 0.01 nM to 2.2 nM. In embodiments, the antibody region binds a target protein with a K_{D} from 0.01 nM to 2.1 nM. In embodiments, the antibody region binds a target protein with a K_{D} from 0.01 nM to 2 nM. In embodiments, the antibody region binds a target protein with a K_{D} from 0.01 nM to 1.9 nM. In embodiments, the antibody region binds a target protein with a K_{D} from 0.01 nM to 1.8 nM. In embodiments, the antibody region binds a target protein with a K_{D} from 0.01 nM to 1.7 nM. In embodiments, the antibody region binds a target protein with a K_{D} from 0.01 nM to 1.6 nM. In embodiments, the antibody region binds a target protein with a K_{D} from 0.01 nM to 1.5 nM. In embodiments, the antibody region binds a target protein with a K_{D} from 0.01 nM to 1.4 nM. In embodiments, the antibody region binds a target protein with a K_{D} from 0.01 nM to 1.3 nM. In embodiments, the antibody region binds a target protein with a K_{D} from 0.01 nM to 1.2 nM. In embodiments, the antibody region binds a target protein with a K_{D} from 0.01 nM to 1.1 nM. In embodiments, the antibody region binds a target protein with a K_{D} from 0.01 nM to 0.9 nM. In embodiments, the antibody region binds a target protein with a K_{D} from 0.01 nM to 0.8 nM. In embodiments, the antibody region binds a target protein with a K_{D} from 0.01 nM to 0.7 nM. In embodiments, the antibody region binds a target protein with a K_{D} from 0.01 nM to 0.6 nM. In embodiments, the antibody region binds a target protein with a K_{D} from 0.01 nM to 0.5 nM. In embodiments, the antibody region binds a target protein with a K_{D} from 0.01 nM to 0.4 nM. In embodiments, the antibody region binds a target protein with a K_{D} from 0.01 nM to 0.3 nM. In embodiments, the antibody region binds a target protein with a K_{D} from 0.01 nM to 0.2 nM. In embodiments, the antibody region binds a target protein with a K_{D} from 0.01 nM to 0.1 nM. In embodiments, the antibody region binds a target protein with a K_{D} of 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 nM.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a Treg cell). Any transmembrane domain capable of anchoring the proteins provided herein including embodiments thereof are contemplated. Non-limiting examples of transmembrane domains include the transmembrane domains of CD28, CD8, CD4 or CD3-zeta. In embodiments, the transmembrane domain is a CD4 transmembrane domain.

In embodiments, the transmembrane domain is a CD28 transmembrane domain. The term “CD28 transmembrane domain” as provided herein includes any of the recombinant or naturally-occurring forms of the transmembrane domain of CD28, or variants or homologs (or functional fragments thereof) thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI:340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof (or functional fragments thereof) that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI:225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof (or functional fragments thereof) that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI:303522473, homolog or functional fragment thereof.

In embodiments, the transmembrane domain is a CD3-zeta (also known as CD247) transmembrane domain. The term “CD3-zeta transmembrane domain” as provided herein includes any of the recombinant or naturally-occurring forms of the transmembrane domain of CD3-zeta, or variants or homologs thereof (or functional fragments thereof) that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, CD3-zeta is the protein as identified by the NCBI sequence reference GI:166362721, homolog or functional fragment thereof.

In embodiments, the chimeric antigen receptor further includes an intracellular signaling domain. An “intracellular signaling domain” as provided herein includes amino acid sequences capable of providing primary signaling in response to binding of an antigen to the antibody region provided herein including embodiments thereof. In embodiments, the signaling of the intracellular signaling domain results in activation of the cell expressing the same. In embodiments, the signaling of the intracellular signaling domain results in proliferation (cell division) of the cell expressing the same. In embodiments, the signaling of the intracellular signaling domain results in expression by said cell of proteins known in the art to characteristic of activated cell (e.g., CTLA-4, PD-1, CD28, CD69). In embodiments, the intracellular cell signaling domain is a CD3 ζ intracellular signaling domain.

In embodiments, the chimeric antigen receptor further includes an intracellular co-stimulatory cell signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the cell expressing the same. In embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB intracellular co-stimulatory signaling domain. In embodiments, the intracellular co-stimulatory signaling domain is an ICOS intracellular co-stimulatory signaling domain. In embodiments, the intracellular co-stimulatory signaling domain is an OX-40 intracellular co-stimulatory signaling domain.

In embodiments, the antibody region includes an Fc domain. In embodiments, the antibody region includes a spacer region. In embodiments, the spacer region is between the transmembrane domain and the antibody region. A “spacer region” as provided herein is a polypeptide connecting the antibody region with the transmembrane domain. In embodiments, the spacer region connects the heavy chain constant region with the transmembrane domain. In embodiments, the spacer region includes an Fc region. In embodiments, the spacer region is an Fc region. Examples of spacer regions contemplated for the compositions provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In embodiments, the spacer region is a hinge region.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof (or functional fragments thereof) that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof (or functional fragments thereof) that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs (or functional fragments thereof) thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-1BB protein, also known as tumor necrosis factor receptor superfamily member 9 (TNFRSF9), Cluster of Differentiation 137 (CD137) and induced by lymphocyte activation (ILA), or variants or homologs thereof (or functional fragments thereof) that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

In embodiments, the reprogrammed Treg cell provided herein including embodiments thereof, further includes a detectable moiety.

In an aspect is provided a tolerogenic dendritic cell generated by the methods provided herein, including embodiments thereof.

In an aspect is provided a reprogrammed Treg generated by the methods provided herein, including embodiments thereof. In embodiments, the reprogrammed Treg is transformed to express FXYD2, NTPDase3, GLP-1R or GPR44 binding domain. In embodiments, the reprogrammed Treg is transformed to express FXYD2 binding domain. In embodiments, the reprogrammed Treg is transformed to express NTPDase binding domain. In embodiments, the reprogrammed Treg is transformed to express GLP-1R binding domain. In embodiments, the reprogrammed Treg is transformed to express GPR44 binding domain.

In embodiments, the reprogrammed Treg provided herein including embodiments thereof is expanded. In embodiments, the reprogrammed Treg is expanded in the presence of interleukin-2 (IL-2).

In embodiments, the reprogrammed Treg is bound to a bispecific antibody. In embodiments, the bispecific antibody includes a GLP-1R or GPR44 targeting domain. In embodiments, the bispecific antibody includes a GLP-1R targeting domain. In embodiments, the bispecific antibody includes a GPR44 targeting domain. In embodiments, the bispecific antibody includes a CD4 targeting domain. In embodiments, the bi-specific antibody targets CD4 and GPR44. In embodiments, the bi-specific antibody targets CD4 and GLP-1R. In embodiments, the antibody region includes a heavy chain constant region (CH) and a light chain constant region (CL). In embodiments, the antibody region includes an Fc domain. In embodiments, the antibody region is a humanized antibody region (e.g. a humanized mouse antibody region).

### Methods of Treatment

Provided herein, inter alia, are methods for treating or preventing immune diseases (e.g. Type 1 diabetes) using the reprogrammed immune cells provided herein including embodiments thereof. Applicants have found that reprogrammed Tregs may be more effective in preventing or inhibiting autoimmune-mediated beta cell destruction as compared to polyclonal Tregs. For example, data from animal models show that 10-100 fold fewer reprogrammed Treg cells are needed to prevent diseases compared to polyclonal Tregs, including T1D. Further, reprogrammed Tregs minimizing the risk for cancer that is possible with the global immune suppression by polyclonal Tregs. Thus, in an aspect is provided a method of treating Type 1 diabetes in a subject in need thereof, the method including administering to the subject a therapeutically effective amount of a reprogrammed Treg cell provided herein including embodiments thereof.

In embodiments, the reprogrammed Treg cells are administered to the patient by re-infusion. In embodiments, a subject who is administered a therapeutically effective amount of the reprogrammed Treg cells provided herein has lower levels of GAD65 or islet cell autoantigen 512 (ICA 512) autoantibodies (anti-ICA 512 autoantibodies) compared to a subject who has Type 1 diabetes who is not treated with the reprogrammed Treg cells. In embodiments, a subject who is administered a therapeutically effective amount of the reprogrammed Treg cells provided herein has lower levels of GAD65 compared to a subject who has Type 1 diabetes who is not treated with the reprogrammed Treg cells. In embodiments, a subject who is administered a therapeutically effective amount of the reprogrammed Treg cells provided herein has lower levels of anti-ICA 512 autoantibodies compared to a subject who has Type 1 diabetes who is not treated with the reprogrammed Treg cells.

In embodiments, the reprogrammed Treg cells are co-administered with an immunosuppressant. In embodiments, the immunosuppressant is abatacept, belatacept, rituximab, cyclosporine, anti-thymocte globulin (ATG), GM-CSF, or sirolimus. In embodiments, the immunosuppressant is abatacept. In embodiments, the immunosuppressant is belatacept. In embodiments, the immunosuppressant is rituximab. In embodiments, the immunosuppressant is cyclosporine. In embodiments, the immunosuppressant is anti-thymocte globulin (ATG). In embodiments, the immunosuppressant is GM-CSF sirolimus. In embodiments, the immunosuppressant is sirolimus.

In another aspect is provided a method of treating or preventing Type 1 diabetes in a subject in need thereof, the method including: (a) contacting a tolerogenic dendritic cell (tolDC) with an islet extracellular vesicle (EV), thereby producing an antigen-loaded tolDC; (b) contacting a T regulatory (Treg) cell with the antigen-loaded tolDC, thereby generating a reprogrammed Treg cell; and administering a therapeutically effective amount of the reprogrammed Treg cell to the subject, thereby treating or preventing Type 1 diabetes in the subject.

In embodiments, the tolDC is produced by contacting a dendritic cell (DC) with interleukin-10 (IL-10). In embodiments, the Treg cell is obtained from a subject with Type 1 diabetes. In embodiments, the DC is obtained from a subject with Type 1 diabetes. In embodiments, the Treg cell and said CD are obtained from the same subject. In embodiments, the islet EV is obtained from a donor who does not have Type 1 diabetes.

In embodiments, step (a) further includes expanding the tolDC. In embodiments, step (b) further includes expanding the reprogrammed Treg cell. In embodiments, the reprogrammed Treg cell is expanded in the presence of IL-2.

In embodiments, the reprogrammed Treg express higher levels of an immunosuppressive cytokine compared to a polyclonal Treg. In embodiments, the immunosuppressive cytokine is IL-10 and/or tumor growth factor-β (TGF-β). In embodiments, the reprogrammed Treg cell secretes lower levels of a proinflammatory cytokine compared to a polyclonal Treg. In embodiments, the proinflammatory cytokine is interferon-γ (IFN-γ), tumor necrosis factor-α (TNF-α), interleukin-4 (IL-4), and/or interleukin-6 (IL-6).

In an aspect is provided a method of treating or preventing type 1 diabetes in a subject in need thereof, the method including administering to the subject a therapeutically effective amount of an autologous regulatory T cell; thereby treating or preventing type 1 diabetes in the subject. For the methods provided herein, in embodiments the autologous regulatory T cell can be formed by a method including: (a) expanding a regulatory T cell from the subject or a donor in vitro, thereby forming an expanded regulatory T cell; and (b) contacting the expanded regulatory T cell with an autologous antigen presenting cell; thereby forming the autologous regulatory T cell. In embodiments, the method includes expanding a regulatory T cell from the subject. In embodiments, the method includes expanding a regulatory T cell from a donor.

For the methods provided herein, in embodiments, the autologous regulatory T cell is formed by a method including: (a) expanding a regulatory T cell from the subject or a donor in vitro, thereby forming an expanded regulatory T cell; (b) contacting an autologous antigen presenting cell with an islet-derived extracellular vesicle in vitro to form an activated autologous antigen presenting cell; and (c) contacting the expanded regulatory T cell with the activated autologous antigen presenting cell; thereby forming the autologous regulatory T cell. In embodiments, the autologous antigen presenting cell includes a plurality of different autologous antigen presenting cells. In embodiments, the regulatory T cell is expanded from the subject. In embodiments, the regulatory T cell is expanded from the donor. In embodiments, the regulatory T cell is isolated from a biological sample of the subject or the donor. In embodiments, the antigen presenting cell is a dendritic cell.

### P Embodiments

P Embodiment 1. A method of generating tolerogenic dendritic cells (tolDCs) comprising contacting islet extracellular vesicles (EVs) with CD14+ monocytes, wherein said contacting results in tolDCs.

P Embodiment 2. The method of embodiment 1, wherein said CD14+ monocytes are obtained from a subject with Type 1 diabetes.

P Embodiment 3. The method of embodiment 1 or 2, wherein said contacting is performed in the presence of interleukin-10 (IL-10).

P Embodiment 4. A tolerogenic dendritic cell generated by any of the methods of claims 1 to 3.

P Embodiment 5. A method of generating reprogrammed T-regulatory cells (Tregs) comprising contacting isolated polyclonal Tregs with the tolerogenic dendritic cell of claim 4, wherein said contacting results in Tregs expressing CD4+, CD25+ and FoxP3.

P Embodiment 6. The method of embodiment 5, wherein said reprogrammed Tregs secrete higher levels of an immunosuppressive cytokine compared to polyclonal Tregs.

P Embodiment 7. The method of embodiment 6, wherein said immunosuppressive cytokine is IL-10 or TGF-beta.

P Embodiment 8. The method of embodiment 5, wherein said reprogrammed Tregs secrete lower levels of a proinflammatory cytokine.

P Embodiment 9. The method of claim 12, wherein said proinflammatory cytokine is interferon-gamma (IFN-gamma), tumor necrosis factor alpha (TNF-alpha), interleukin-4 (IL-4), or interleukin-6 (IL-6).

P Embodiment 10. The method of embodiment 8, wherein said reprogrammed Tregs increase suppression of CD4+ and CD8+ T cell proliferation compared to polyclonal Tregs.

P Embodiment 11. A reprogrammed Treg generated by any of the methods of embodiments 5-10.

P Embodiment 12. The reprogrammed Treg of embodiment 11, transformed to express FXYD2, NTPDase3, GLP-1R or GPR44 binding domain.

P Embodiment 13. The reprogrammed Treg of embodiment 12, wherein said reprogrammed Treg is expanded.

P Embodiment 14. The reprogrammed Treg of embodiment 13, expanded in the presence of interleukin-2 (IL-2).

P Embodiment 15. The reprogrammed Treg of embodiment 11, comprising a chimeric antigen receptor (CAR).

P Embodiment 16. The reprogrammed Treg of embodiment 15, wherein the CAR is a bispecific CAR.

P Embodiment 17. The reprogrammed Treg of embodiment 15 or 16, wherein the CAR comprises a humanized antibody sequence.

P Embodiment 18. The reprogrammed Treg of any one of embodiments 15-17, wherein the CAR targets beta cells.

P Embodiment 19. The reprogrammed Treg of any one of embodiments 15-18, wherein the CAR targets one or more of FXYD2, NTPDase3, GLP-1R or GPR44.

P Embodiment 20. The reprogrammed Treg of embodiment 11, bound to a bispecific antibody.

P Embodiment 21. The reprogrammed Treg of embodiment 20, wherein the bispecific antibody comprises a GPR44 binding domain.

P Embodiment 22. The reprogrammed Treg of embodiment 20 or 21, wherein the bispecific antibody comprises a CD4 binding domain.

P Embodiment 23. The reprogrammed Treg of any one of embodiments 20-22, wherein the bispecific antibody comprises two or more of a FXYD2, NTPDase3. GPR44 or CD4 binding domain.

P Embodiment 24. A method of treating Type 1 diabetes to a subject in need, said method comprising administering the reprogrammed Treg of embodiment 11 to said subject.

P Embodiment 25. A method of treating or preventing type 1 diabetes in a subject in need thereof, the method comprising administering to the subject a therapeutically effective amount of an autologous regulatory T cell; thereby treating or preventing type 1 diabetes in the subject.

P Embodiment 26. The method of embodiment 25, comprising administering to the subject a therapeutically effective amount of a plurality of autologous regulatory T cells.

P Embodiment 27. The method of any one of embodiments 25-27, wherein the autologous regulatory T cell is formed by a method comprising: (a) expanding a regulatory T cell from the subject or a donor in vitro, thereby forming an expanded regulatory T cell; and (b) contacting the expanded regulatory T cell with an autologous antigen presenting cell in vitro; thereby forming the autologous regulatory T cell.

P Embodiment 28. The method of any one of embodiments 25-27, wherein the autologous regulatory T cell is formed by a method comprising: (a) expanding a regulatory T cell from the subject or a donor in vitro, thereby forming an expanded regulatory T cell; (b) contacting an autologous antigen presenting cell with an islet-derived extracellular vesicle in vitro to form an activated autologous antigen presenting cell; and (c) contacting the expanded regulatory T cell with the activated autologous antigen presenting cell in vitro; thereby forming the autologous regulatory T cell.

P Embodiment 29. The method of any one of embodiments 25-28, wherein the autologous antigen presenting cell comprises a plurality of different autologous antigen presenting cells.

P Embodiment 30. The method of any one of embodiments 25-29, wherein the regulatory T cell is expanded from the subject.

P Embodiment 31. The method of any one of embodiments 25-29, wherein the regulatory T cell is expanded from the donor.

P Embodiment 32. The method of any one of embodiments 25-29, wherein the regulatory T cell is isolated from a biological sample of the subject or the donor.

P Embodiment 33. The method of any one of embodiments 27-33, wherein the antigen presenting cell is a dendritic cell.

### Embodiments

Embodiment 1. A method of generating a reprogrammed T regulatory (Treg) cell, the method comprising: (a) contacting a dendritic cell (DC) with interleukin-10 (IL-10), thereby producing a tolerogenic dendritic cell (tolDC); (b) contacting said tolDC with an islet extracellular vesicle (EV), thereby producing an antigen-loaded tolDC; and (c) contacting a Treg cell with said antigen-loaded tolDC, thereby producing said reprogrammed Treg cell.

Embodiment 2. The method of embodiment 1, wherein said Treg cell is obtained from a subject with Type 1 diabetes.

Embodiment 3. The method of embodiment 1 or 2, wherein said DC is obtained from a subject with Type 1 diabetes.

Embodiment 4. The method of any one of embodiments 1-3, wherein said Treg cell and said DC are obtained from the same subject.

Embodiment 5. The method of any one of embodiments 1-4, wherein said islet EV is obtained from a donor who does not have Type 1 diabetes.

Embodiment 6. The method of any one of embodiments 1-5, wherein step (a) further comprises expanding said tolDC.

Embodiment 7. The method of any one of embodiments 1-6, wherein step (b) further comprises expanding said antigen-loaded tolDC.

Embodiment 8. The method of any one of embodiments 1-7, wherein step (c) further comprises expanding said reprogrammed Treg cell.

Embodiment 9. The method of embodiment 8, wherein said reprogrammed Treg cell is expanded in the presence of IL-2.

Embodiment 10. The method of any one of embodiments 1-9, wherein said reprogrammed Treg cell express higher levels of an immunosuppressive cytokine compared to a polyclonal Treg cell.

Embodiment 11. The method of embodiment 10, wherein said immunosuppressive cytokine is IL-10 and/or tumor growth factor-β (TGF-β).

Embodiment 12. The method of any one of embodiments 1-11, wherein said reprogrammed Treg cell secretes lower levels of a proinflammatory cytokine compared to a polyclonal Treg.

Embodiment 13. The method of claim 12, wherein said proinflammatory cytokine is interferon-γ (IFN-γ), tumor necrosis factor-α (TNF-α), interleukin-4 (IL-4), and/or interleukin-6 (IL-6).

Embodiment 14. The method of any one of embodiments 1-13, wherein said reprogrammed Treg cell increases suppression of CD4+ and CD8+ T cell proliferation compared to a polyclonal Treg cell.

Embodiment 15. A reprogrammed Treg cell made by the method of any one of embodiments 1-14.

Embodiment 16. The reprogrammed Treg cell of embodiment 15, wherein said reprogrammed Treg cell express higher levels of an immunosuppressive cytokine compared to a polyclonal Treg cell.

Embodiment 17. The reprogrammed Treg cell of embodiment 16, wherein said immunosuppressive cytokine is IL-10 and/or tumor growth factor-β (TGF-β).

Embodiment 18. The reprogrammed Treg cell of any one of embodiments 15-17, wherein said reprogrammed Treg cell secretes lower levels of a proinflammatory cytokine compared to a polyclonal Treg cell.

Embodiment 19. The reprogrammed Treg cell of embodiment 18, wherein said proinflammatory cytokine is interferon-γ (IFN-γ), tumor necrosis factor-α (TNF-α), interleukin-4 (IL-4), and/or interleukin-6 (IL-6).

Embodiment 20. The reprogrammed Treg cell of any one of embodiments 15-19, wherein said reprogrammed Treg cell targets a protein expressed on the surface of a pancreatic beta cell.

Embodiment 21. The reprogrammed Treg cell of embodiment 20, wherein said protein is Sodium/potassium-transporting ATPase subunit gamma (FXYD2), NTPDase3, Glucagon-like peptide-1 receptor (GLP-1R) or GPR44.

Embodiment 22. The reprogrammed Treg cell of any one of embodiments 15-21, comprising a chimeric antigen receptor (CAR).

Embodiment 23. The reprogrammed Treg cell of embodiment 22, wherein said CAR comprises an (i) antibody region comprising a FXYD2, NTPDase3, GLP-1R or GPR44 binding domain; and (ii) a transmembrane binding domain.

Embodiment 24. The reprogrammed Treg cell of any one of embodiments 15-23, further comprising a detectable moiety.

Embodiment 25. A method of treating Type 1 diabetes in a subject in need thereof, said method comprising administering to said subject a therapeutically effective amount of the reprogrammed Treg cell of any one of embodiments 15-24.

Embodiment 26. A method of treating or preventing Type 1 diabetes in a subject in need thereof, the method comprising: (a) contacting a tolerogenic dendritic cell (tolDC) with an islet extracellular vesicle (EV), thereby producing an antigen-loaded tolDC; (b) contacting a T regulatory (Treg) cell with said antigen-loaded tolDC, thereby generating a reprogrammed Treg cell; and (c) administering a therapeutically effective amount of said reprogrammed Treg cell to said subject, thereby treating or preventing Type 1 diabetes in the subject.

Embodiment 27. The method of embodiment 26, wherein said tolDC is produced by contacting a dendritic cell (DC) with interleukin-10 (IL-10).

Embodiment 28. The method of embodiment 26 or 27, wherein said Treg cell is obtained from a subject with Type 1 diabetes.

Embodiment 29. The method of any one of embodiments 26-28, wherein said DC is obtained from a subject with Type 1 diabetes.

Embodiment 30. The method of any one of embodiments 26-29, wherein said Treg cell and said CD are obtained from the same subject.

Embodiment 31. The method of any one of embodiments 26-30, wherein said islet EV is obtained from a donor who does not have Type 1 diabetes.

Embodiment 32. The method of any one of embodiments 26-31, wherein step (a) further comprises expanding said tolDC.

Embodiment 33. The method of any one of embodiments 26-32, wherein step (a) further comprises expanding said antigen-loaded tolDC.

Embodiment 34. The method of any one of embodiments 26-32, wherein step (a) further comprises expanding said antigen-loaded tolDC.

Embodiment 35. The method of embodiment 34, wherein said reprogrammed Treg cell is expanded in the presence of IL-2.

Embodiment 36. The method of any one of embodiments 26-35, wherein said reprogrammed Treg express higher levels of an immunosuppressive cytokine compared to a polyclonal Treg.

Embodiment 37. The method of embodiment 36, wherein said immunosuppressive cytokine is IL-10 and/or tumor growth factor-β (TGF-β).

Embodiment 38. The method of any one of embodiments 26-37 wherein said reprogrammed Treg cell secretes lower levels of a proinflammatory cytokine compared to a polyclonal Treg.

Embodiment 39. The method of embodiment 38, wherein said proinflammatory cytokine is interferon-γ (IFN-γ), tumor necrosis factor-α (TNF-α), interleukin-4 (IL-4), and/or interleukin-6 (IL-6).

### EXAMPLES

One skilled in the art would understand that descriptions of the methods, reprogrammed cells, and compositions described herein is for the sole purpose of illustration, and that the present disclosure is not limited by this illustration.

### Introduction to Exemplary Studies

Type one diabetes (T1D) affects 2 million people in Europe and North America and is rising. In T1D, autoimmune activation leads to beta-cell destruction through interactions among different types of activated T cells (e.g., cytotoxic T cells, natural killer (NK) T cells, and memory T cells), and antigen-presenting cells (dendritic cells, macrophages, and B cells). Normally T regulatory cells (Tregs), suppress these destructive immune processes. However, Tregs are either dysfunctional or scarce in T1D patients (1). Enhancing Tregs' functional capacity in T1D patients is one mechanism to halt or prevent beta-cell destruction and diabetes. Treatment with polyclonal Tregs while well-tolerated (2, 3), did not preserve C-peptide production in newly diagnosed T1D in phase 2 clinical trial (NCT02691247). Preclinical studies show that Tregs that are continuously stimulated by antigens expressed in the target organ is better at modulating organ-specific autoimmune diseases. Identification of new methods to re-program/educate autologous Tregs to become antigen-specific, long-lasting and effective in suppressing dysfunctional immune responses, may prove useful for quelling T1D-associated beta-cell autoimmune attack.

The development of autoimmunity to self-antigens, such as glutamic acid decarboxylase (GAD) and insulin/proinsulin, is a hallmark for high risk for the destruction of beta-cell mass and the development of T1D. Pre-clinical studies suggest that antigen-specific Tregs (aTreg) to these self-antigens are more effective in controlling autoimmune-mediated beta-cell destruction compared to polyclonal Tregs, with animal models showing 10-100 fold fewer cells required to prevent disease (4-8). However, to date, aTreg therapy to treat T1D has not been studied in the clinical setting. Although potential target antigens have already been defined in T1D (9), a major challenge lies in the identification of all the potential target antigens involved in the pathogenesis of all patients with T1D. To this end, a process for Treg education/programming that does not require prior identification of the specific autoantigens unique to each patient, but rather, utilizes the isolated normal islet extracellular vesicles (that likely contain all islet autoantigens) for Treg education/programming is provided.

One challenge of many cell-based therapies is the trafficking of the therapeutic cells to the disease site: in T1D, islet microenvironment. Therefore, in addition to programming/educating the Tregs to become antigen-specific and more potent, aTreg will also be engineered to express beta-cell specific targeting ligand to direct the autologous aTregs to the beta-cell environment within the pancreas after infusion. Homing of aTregs to the disease site has the potential to augment the aTregs' treatment efficacy. Utilizing chimeric antigen receptors (CAR) T therapy from the field of immune-oncology, aTregs educated with dendritic cells (DC) pulsed with the islet extracellular vesicles (EV) will be transduced with the lentiviral vector containing transgene sequence expressing a binding domain for GLP-1R or GPR44. GPR44 is a receptor for prostaglandin D2 (PGD2) that has recently been shown to be restricted to beta-cells in the human pancreas (10) and has been successfully targeted for visualization of beta-cells in vivo in an immune-deficient mouse model of human islet transplantation (11). In silico humanization of antibody sequences to be used as binding domains for the GPR44 receptor or GLP-1 receptor surface molecules has been completed. These antibody sequences may be used for manufacturing GPR44-CAR-aTregs or GLP-1-CAR-aTregs, as well as for the production of a bi-specific antibody with two distinct binding sites (i.e. binding sites for GPR44 or GLP-1 and CD4).

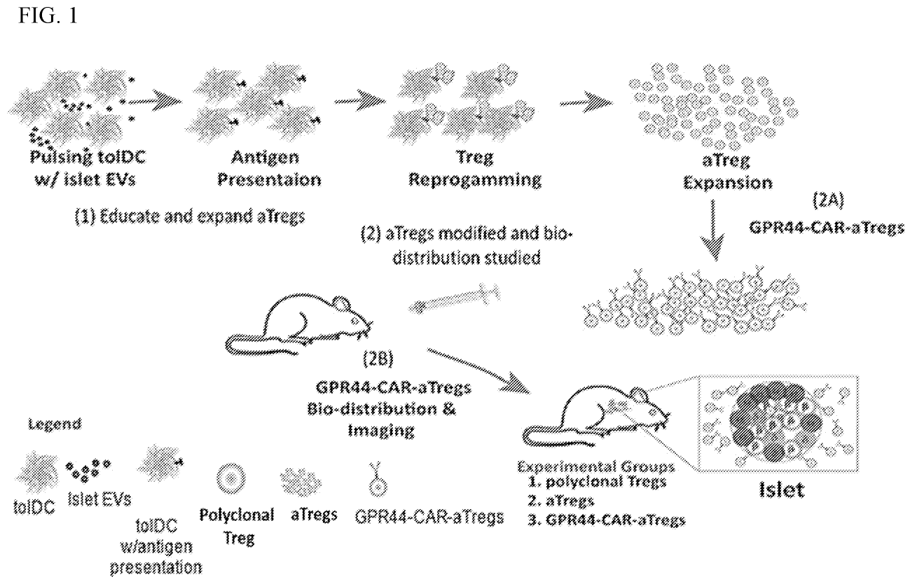

Studies described herein identify effective methods to generate beta-cell targeting aTregs that can migrate to islet microenvironment and curtail the immune responses that lead to beta-cell destruction. These methods are achieved via the combination of Treg reprogramming, in silico modeling and ex vivo genetic modification of these cells to generate CAR-aTregs, which are tested in vivo to demonstrate safety and bio-distribution of GPR44-CAR-aTegs or GLP-1-CAR-aTregs as compared to aTregs alone and polyclonal Tregs, as shown in the schematic of FIG. 1. Collectively, generated data inform a phase 1/2 clinical trial to a) establish safety and preliminary efficacy of beta-cell targeting aTreg for the treatment of recently diagnosed T1D patients with residual beta-cell function and, b) demonstrate the clinical utility of beta-cell targeting aTreg infusion after islet transplantation. The newly engineered cells have the potential to modulate the immune system in a site-specific manner without compromising immune responses to pathogens. This increases the safety of the immunomodulatory regimen and increases the potential for achieve the meaningful and efficacious clinical outcomes in T1D. Targeting aTreg based intervention are applicable to other autoimmune diseases.

### Example 1: Generation of T1D Autologous aTregs by Reprogramming Tregs with Antigen-Presenting Cells (APCs) Ex Vivo

Recently, the safety and promising outcomes of ex-vivo expanded autologous Treg therapy in patients with T1D was reported. Children with new-onset T1D (within 2 months of diagnosis) showed prolonged survival of functional beta-cells and reduced exogenous insulin use in the majority of patients. In fact, two patients were free of exogenous insulin at one year (2, 3). The safety and feasibility of administration of polyclonal Tregs in 14 adults with T1D within 14-104 weeks of diagnosis was further reported. Approximately 25% of infused Tregs persisted in the circulation after a year, however, metabolic data including insulin-use, HbA1c levels, and C-peptide remained unchanged in the majority of patients. Except for the significant reduction in NK cells early after Treg treatment, neither effector T cells nor GAD65 or islet cell autoantigen 512 (ICA 512) autoantibodies were significantly modified during the course of the study. Furthermore, a phase 2 clinical trial to address the efficacy of polyclonal Treg treatment in 112 newly-diagnosed adolescent T1D patients (NCT02691247) did not show clinical activity. Notwithstanding these mixed reports, in pre-clinical studies, Tregs that are continuously stimulated by specific-antigens expressed in the target organ are better at modulating organ-specific autoimmune diseases (4, 5, 12, 13).

Polyclonal Tregs Significantly Delay GvHD and Diabetes in Mice Models

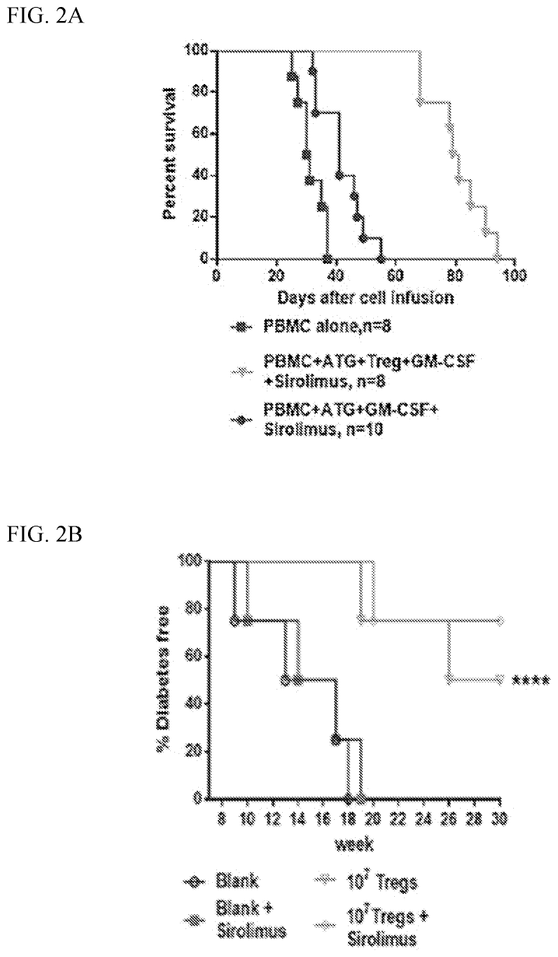

The methods described herein were used to isolate Tregs from diabetic patients and healthy donors and expand them up to 2,000-fold in the laboratory (average: ˜600-fold). After expansion, the infusion of polyclonal Tregs improved the effectiveness of ATG, GM-CSF, and sirolimus in prolonging the survival in graft vs. host disease in NSG mice (FIG. 2A). More importantly, polyclonal Treg/Sirolimus combination therapy blocked the development of T1D in the non-obese diabetic (NOD) mouse models (FIG. 2B).

Islet EV Express Autoantigens

Human islet-derived EVs were isolated from non-diabetic islet cell culture supernatant. The supernatant was freshly centrifuged at 300×g for 10 min, then at 12,500×g for 20 min, and finally at 100,000×g for 70 min. EVs were then resuspended in PBS and stored at −80° C. EV concentration was determined with the Micro BCA Protein Assay kit (Thermo Fisher) according to the manufacturer's instructions. EV particle number was determined by nanosight instrument.

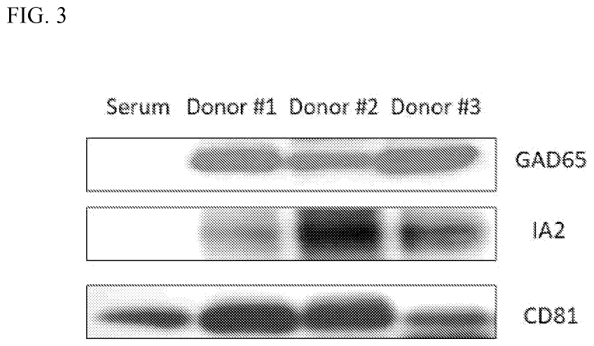

The islet EVs isolated from the culture medium of healthy human islets were grown in vitro and were subjected to an immunoblot assay. Protein extracts from islet EVs were analyzed for the autoantigens GAD65 and islet antigen 2 (IA2). Autoantigens, GAD65 and IA2, were present in the islet EVs (FIG. 3) but not in the serum. An EV specific marker, CD81, was detected in both islet EVs and EVs isolated from serum.

Generation of tolDC with Interleukin (IL) 10

Mature DC (mDC) and tolDC (DC-10) were generated from CD14 monocytes of patients with T1D, which were isolated by magnetic bead separation from leukapheresis product, and induced to monocyte-derived dendritic cells in RPMI-1640 medium with 7% fetal bovine serum (FBS) for 6 days in the presence of Granulocyte-Macrophage Colony Stimulating Factor (GM-CSF), IL-4 and with or without, IL-10, respectively.

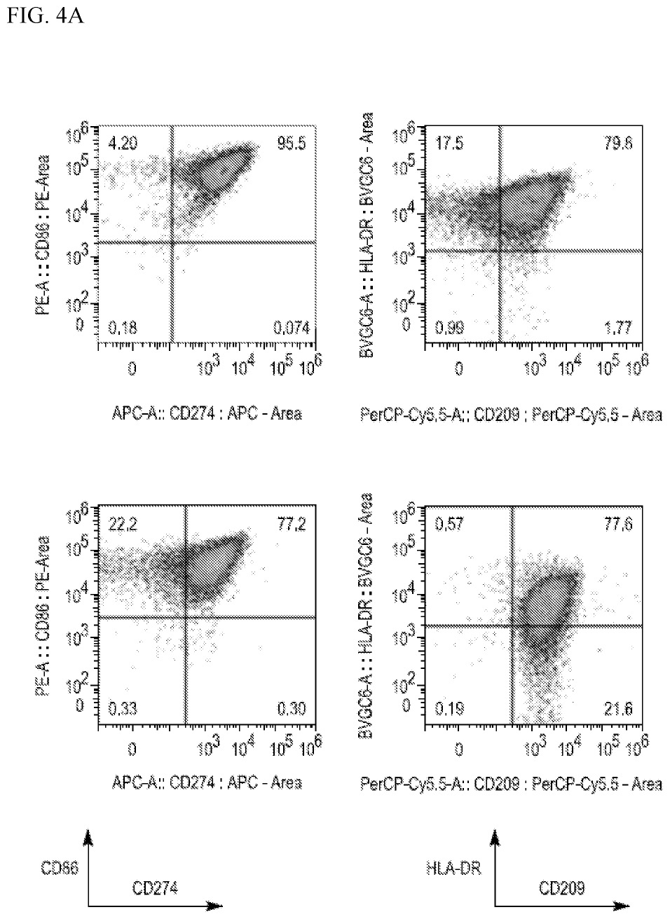

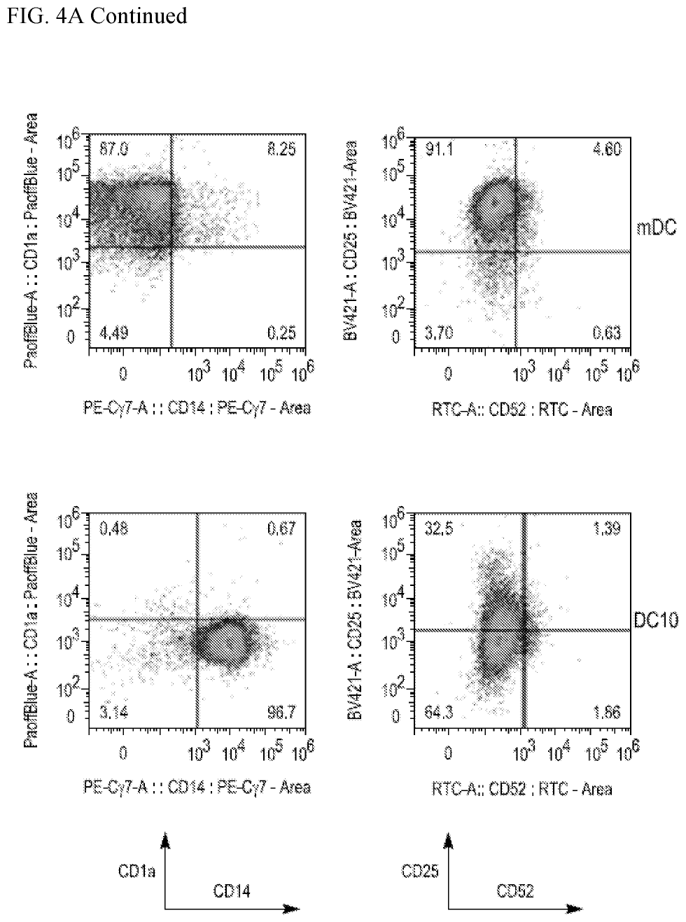

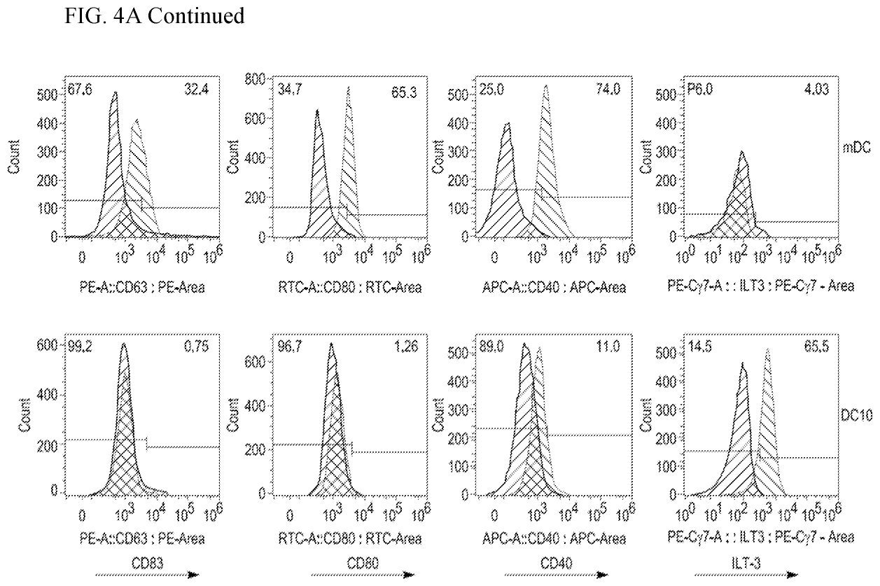

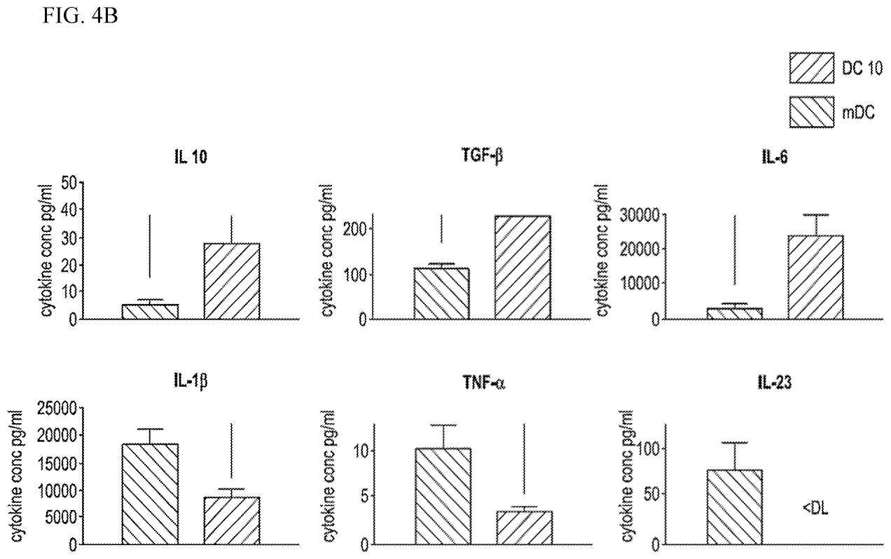

After maturation with a cytokine cocktail comprising GM-CSF, IL-6. IL-1β, TNFα and PGE-2, the phenotype and cytokines profiles of DC-10 and mDC were analyzed and compared. Fluorescence-activated cell sorting (FACS) analysis showed that DC-10 expressed lower levels of CD86, HLA-DR, CD1a, CD80, CD83, CD40, and higher levels of CD14, immunoglobulin-like transcript (ILT-3) than mDCs (FIG. 4A).

In addition, DC-10 produced more IL-10, transforming growth factor-beta (TGF-beta) and IL-6, but less IL-1beta, tumor necrosis factor-α (TNF-α) and IL-23 compared to mDC (FIG. 4B). Therefore, DC-10 shows superior tolerogenic features (16) and a more immature phenotype (17) than mDC. DC-10 were utilized to educate polyclonal Tregs in all preliminary studies.

Autologous aTregs Educated with tolDC Pulsed with Cultured Normal Islet EVs Display aTreg Phenotype

CD4⁺CD25⁺CD127^{low} Tregs of patients with T1D were educated with tolDC of the respective patient loaded with islet EVs from healthy donors and expanded for 3 weeks. The Tregs were educated with the antigen-loaded tolDC at 5:1 (Treg:DC) ratio for two rounds and then expanded by CD3/CD28/CD2 T cell activator in X-VIVO 20 media in the presence of human IL-2. Polyclonal Tregs were generated by expanding Tregs by CD3/CD28/CD2 T cell activator with human IL-2 in X-VIVO 20 media for up to 3 weeks.

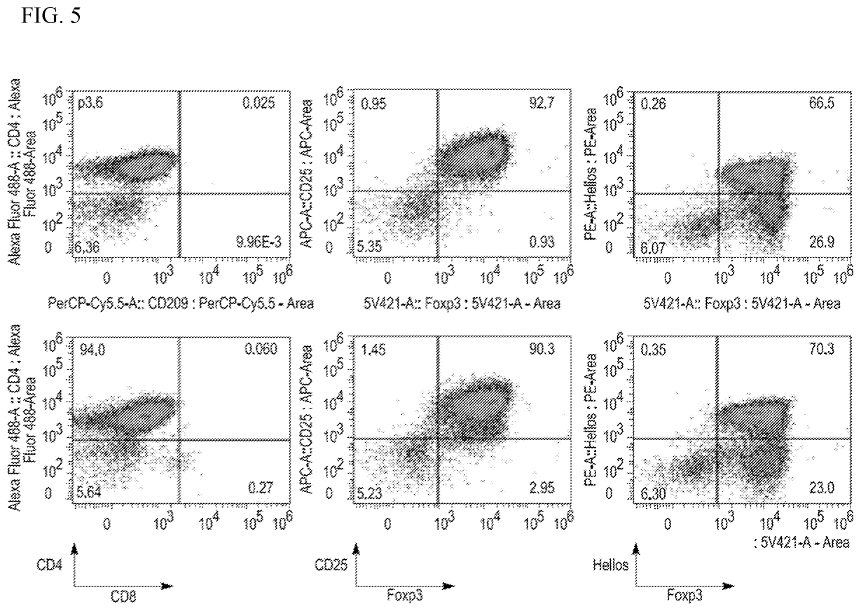

Flow cytometry analysis confirmed Treg phenotypic expression of CD4+, CD25+ and forkhead box P3 (Foxp3) on expanded Tregs (FIG. 5). Both aTregs and polyclonal Tregs expressed similar level of Foxp3 and Helios.

Autologous aTregs Secrete Higher Levels of Immunosuppressive Cytokines

A bead-based immunoassay was used to determine the cytokine profile of aTregs and polyclonal Tregs after expansion. Briefly, 5×10⁵ aTregs and polyclonal Tregs were stimulated in X-VIVO 20 media+10% Human AB serum in the presence of CD3/CD28/CD2 T cell activators for 24 hrs. Culture supernatants were collected after the stimulation.

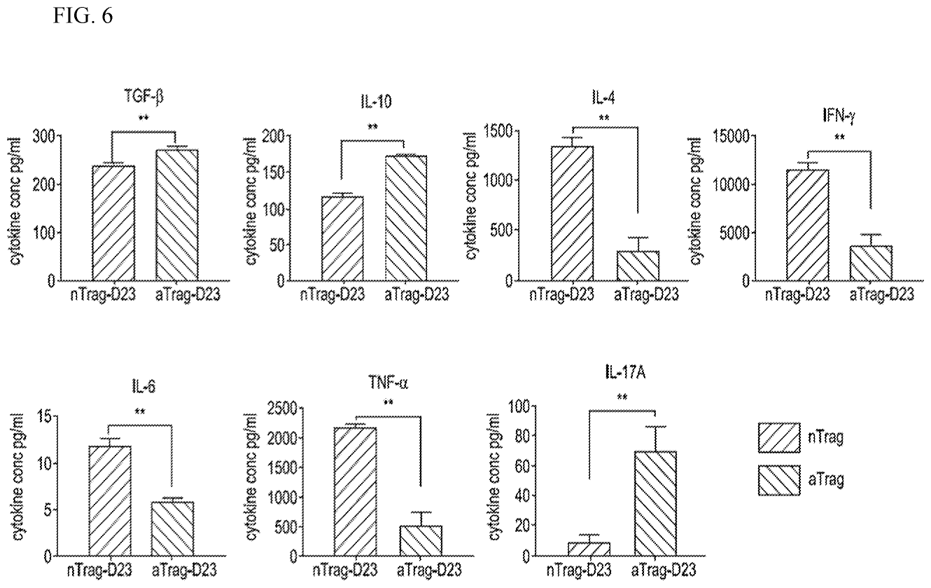

The culture supernatant from the expanded polyclonal Tregs (nTregs) and aTregs from T1D patients was then analyzed by LEGENDplex kits (Biolegend) according to the manufacturer's instructions. Interferon γ (IFN γ), TNF-α, IL-4, IL-6, IL-17, IL-10 and TGF-beta secretion were detected (FIG. 6). aTregs secreted higher levels of immunosuppressive cytokines, IL-10 and TGF-beta than polyclonal Tregs. Further, aTregs secreted lower levels of proinflammatory cytokines, (IFN γ), TNF-α, IL-4, and IL-6.

Autologous aTregs Show Enhanced Suppressive Activity

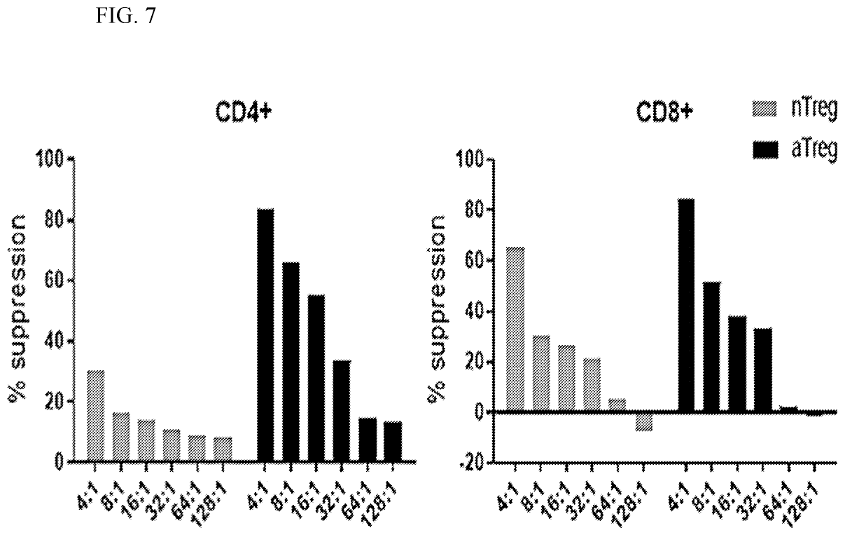

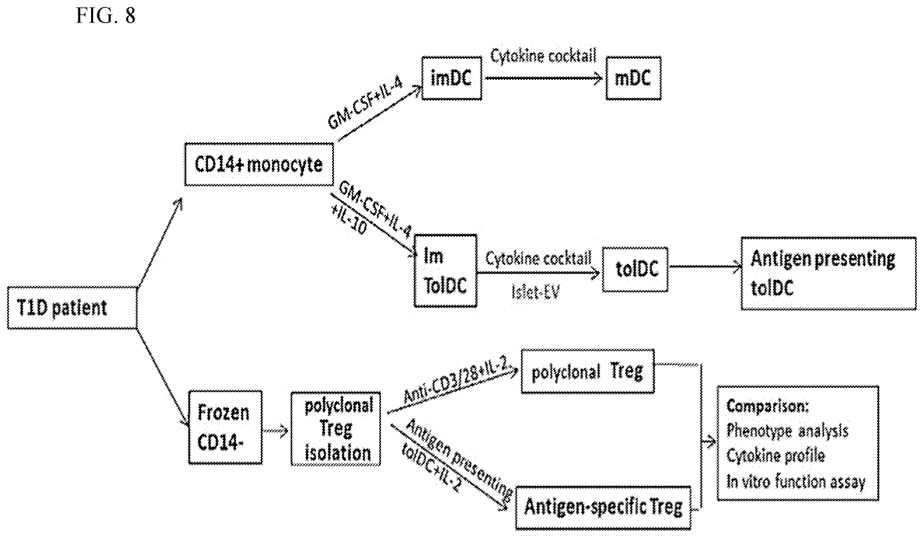

Antigen-specific suppression assay was used to determine proliferation of Teff cells co-cultured with aTregs or polyclonal Tregs. CFSE labeled PBMCs from patients with T1D were co-cultured with aTregs of the respective individual or polyclonal Tregs under recall antigen (Pentacel) (18) and islet-derived EVs for 7 days. A range of Treg to PBMC ratios (1:1, 1:2, 1:4, 1:8, 1:16, 1:32, 1:64, 1:128 and 0:1) are tested. FACS analysis showed the increased suppressive activity of aTregs vs polyclonal Tregs in inhibiting CD4⁺ and CD8⁺ T cell proliferation (FIG. 7).

### Example 2: Generation of Autologous aTregs Designed to Traffic to the Islet Microenvironment

To enhance immune modulation in an organ-specific manner, the delivery of aTregs to the pancreatic microenvironment is essential. Thus, exploiting beta-cell-specific markers to traffic aTregs to the islet microenvironment may improve the effectiveness of aTregs. To that end, through in silico modeling sequences to the GPR44 receptor domain are extracted for transduction of reprogrammed aTregs into CAR-aTregs targeting GPR44. GPR44 is also designated as the prostaglandin D2 (PGD2) receptor 2 that restrict to beta cells (11). Several engineered CAR-Tregs reversed multiple sclerosis and colitis (22-24) in mice models. Also, in a graft vs host disease (GVHD) setting, fully humanized HLA-A2-CAR Treg prevented HLA-A2+ skin graft rejection by HLA-A2-PBMCs (25, 26). Moreover, CAR-Tregs specific to insulin reversed T1D in NOD mice (27). Thus, aTregs that migrate to the pancreas could be potent regulators that can effectively modulate immune responses and can play a critical role in mitigating the development of T1D. Autologous aTregs are developed that traffic to the beta-cells.

Develop Antigen-Specific GPR44-CAR-aTregs that Traffic to the Pancreatic Environment

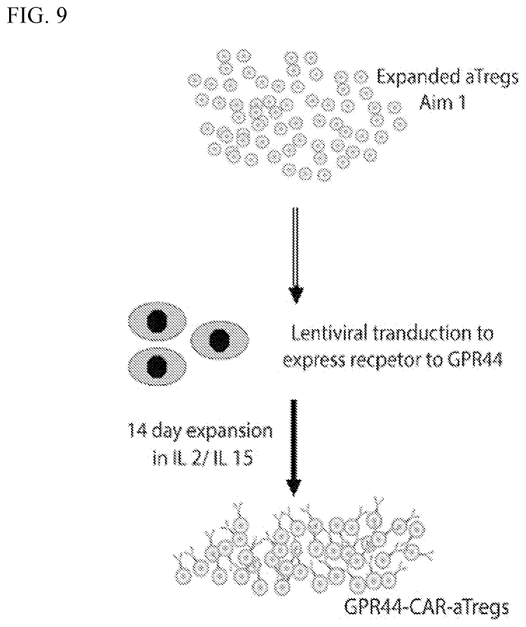

aTregs educated with DC pulsed with the islet EVs as described in herein are transduced with the lentiviral vector containing the transgene sequence expressing a GPR44 binding domain. aTregs are transduced with third-generation lentivector containing transgene sequence for 1) antibody single-chain variable fragment (scFV) extracellular domain that binds to GPR44 receptor (see below) and 2) CAR backbone containing: IgG4Fc (EQ) linker, CD4 transmembrane domain (CD4tm), and the co-stimulatory domain, 4-1BB (CD137) and CD3ζ (cytoplasmic signaling domains), as well as the T2A ribosomal skip driven by the human EF1α promoter (EF1p) (28). This set of CAR backbone domains has been successfully used to generate CD19-CAR-T cells and uses four plasmids to generate the viral particles which reduce the chance of recombination and ensures safety by separating viral genetic components. The four plasmids are then transfected into a producer cell line using lipofectamine transfection reagent to generate the viral particles. aTregs are then spin infected at 31° C. for 1.5 h at 700× g with the viral particles encoding the GPR44 binding-domain. After lentiviral transduction, GPR44-aTregs are expanded for 14 days in the presence of IL-2 and IL-15. The schematic of CAR transduction is shown in FIG. 9.

For the extracellular domain that binds to GPR44 receptor, the murine monoclonal antibody to GPR44 receptor was sequenced by mass spectrometry and computational algorithms were used to humanize the antibody. The gene sequence encoding this antibody is codon optimized to enhance expression and directly synthesized in an expression vector. In addition to expressing and purifying the humanized αGPR44 mAb, the extracellular domain of the GPR44 receptor is expressed and purified and used to characterize the binding affinity of the humanized αGPR44 mAb. Likewise, the thermal stability of the mAb is determined using differential scanning fluorimetry (DSF). If necessary (low affinity and thermal melting point), yeast display is used to affinity and thermal stabilize the mAb. Once similar endpoints (K_{D}<10 nM and T_{M}>65° C.) are achieved, the sequence for the construction of transgene is extracted for the generation of CAR-aTregs.

The potency of GPR44 CAR-aTregs is assessed by 1) bead-based immunoassay to determine the cytokine profile and 2) suppression assay to determine Teff proliferation, and 3) ELISA to determine the Teff cell function described herein.

Examination of Bio-Distribution and In Vivo Imaging of aTregs Expressing GPR44 Binding Domain in a Mice Model

aTregs are most effective in inducing immune tolerance in an organ-specific manner. aTreg cells trafficking to the organ site is one factor that may be the predictive indicator of therapeutic outcome. To accurately assess full potency of engineered aTregs, in vivo targeting of engineered Tregs to the pancreatic microenvironment is essential. ⁸⁹Zr-oxine has been successfully used for labeling and imaging of CAR-T cells in vivo without affecting cell viability and function (29) at COH and ⁸⁹Zr-oxine labeled CAR-T cells were detectable by positron emission tomography (PET) scan for up to 6 days after the infusion.

To determine the bio-distribution and in vivo imaging of the aTregs: 1) nTregs, 2) aTregs, and 3) GPR44-CAR-aTregs, the Tregs are labeled with 0-1.4 MBq of ⁸⁹Zr-oxine/10⁶ cells for 30 min at 37° C. Cell-labeling efficiency is determined as the percentage radioactivity in the Tregs/total radioactivity. Radioactivity retention in Tregs is measured by a γ-counter.

The following assays are performed to ensure that ⁸⁹Zr-oxine labeling does not affect cell viability and Treg function, and to determine the bio-distribution of the engineered aTregs.

To determine the viability of ⁸⁹Zr-oxine labeled engineered Tregs, ⁸⁹Zr-oxine labeled: polyclonal Tregs, aTreg, and GPR44-CAR-aTreg, and unlabeled GPR44-CAR-aTreg are cultured for 1, 2 and 5 days and labeled Tregs is cultured with 0.1% propidium iodide (PI). The incorporation of PI is assessed on a Moxi Flow cassette-based flow cytometer. Unlabeled GPR44-CAR-aTregs are used as controls.

Functional capability of ⁸⁹Zr-oxine labeled engineered aTregs is assayed by stimulating the cells for 4 hrs by plate-bound GPR44 in culture and quantifying immunosuppressive cytokines in culture medium by ELISA: TGF-β, IFNγ, TNF-α, IL-17, and IL-10. Cytokine levels secreted by unlabeled engineered aTregs are used as controls.

Bio-distribution and imaging of ⁸⁹Zr-oxine labeled engineered aTregs is performed by intravenous infusion of ⁸⁹Zr-oxine labeled 2.0×10⁶ polyclonal Tregs, aTregs and GPR44-CAR-aTregs, into 6-8 weeks old NSG mice. Major organs are removed including the pancreas, heart, lung, liver, spleen, stomach, intestines, kidneys, right quadriceps, right femur, left stifle joint and lumbar vertebrae. Radioactivity in the organs is measured using an automated γ-counter. The percentage injected dose (% ID) and % ID/g of each tissue is calculated. PET scans are utilized to detect the labeled aTregs cells in the animals. The trafficking of polyclonal Tregs, aTregs, and GPR44-CAR-aTregs to the pancreas are compared.

### REFERENCES

Hull C M, Peakman M, Tree T I M. Regulatory T cell dysfunction in type 1 diabetes: what's broken and how can we fix it? Diabetologia. 2017; 60(10):1839-50. doi: 10.1007/s00125-017-4377-1. PubMed PMID: 28770318; PMCID: PMC6448885.
Marek-Trzonkowska N, Mysliwiec M, Dobyszuk A, Grabowska M, Techmanska I, Juscinska J, Wujtewicz M A, Witkowski P, Mlynarski W, Balcerska A, Mysliwska J, Trzonkowski P. Administration of CD4+CD25highCD127-regulatory T cells preserves beta-cell function in type 1 diabetes in children. Diabetes Care. 2012; 35(9):1817-20. doi: 10.2337/dcl2-0038. PubMed PMID: 22723342; PMCID: PMC3425004.
Marek-Trzonkowska N, Mysliwiec M, Dobyszuk A, Grabowska M, Derkowska I, Juscinska J, Owczuk R, Szadkowska A, Witkowski P, Mlynarski W, Jarosz-Chobot P, Bossowski A, Siebert J, Trzonkowski P. Therapy of type 1 diabetes with CD4(+)CD25(high)CD127-regulatory T cells prolongs survival of pancreatic islets—results of one year follow-up. Clin Immunol. 2014; 153(1):23-30. doi: 10.1016/j.clim.2014.03.016. PubMed PMID: 24704576.
Tarbell K V, Yamazaki S, Olson K, Toy P, Steinman R M. CD25+CD4+ T cells, expanded with dendritic cells presenting a single autoantigenic peptide, suppress autoimmune diabetes. J Exp Med. 2004; 199(11):1467-77. doi: 10.1084/jem.20040180. PubMed PMID: 15184500; PMCID: PMC2211787.
Wheeler K M, Samy E T, Tung K S. Cutting edge: normal regional lymph node enrichment of antigen-specific regulatory T cells with autoimmune disease-suppressive capacity. J Immunol. 2009; 183(12):7635-8. doi: 10.4049/jimmunol.0804251. PubMed PMID: 19923458; PMCID: PMC2872190.
Tarbell K V, Petit L, Zuo X, Toy P, Luo X, Mqadmi A, Yang H, Suthanthiran M, Mojsov S, Steinman R M. Dendritic cell-expanded, islet-specific CD4+CD25+CD62L+ regulatory T cells restore normoglycemia in diabetic NOD mice. J Exp Med. 2007; 204(1):191-201. doi: 10.1084/jem.20061631. PubMed PMID: 17210729; PMCID: PMC2118426.
Tang Q, Henriksen K J, Bi M, Finger E B, Szot G, Ye J, Masteller E L, McDevitt H, Bonyhadi M, Bluestone J A. In vitro-expanded antigen-specific regulatory T cells suppress autoimmune diabetes. J Exp Med. 2004; 199(11):1455-65. doi: 10.1084/jem.20040139. PubMed PMID: 15184499; PMCID: PMC2211775.
Ozdal O L, Aprikian A G, Begin L R, Behlouli H, Tanguay S. Comparative evaluation of various prostate specific antigen ratios for the early detection of prostate cancer. BJU Int. 2004; 93(7):970-4; discussion 4. doi: 10.1111/j.1464-410X.2003.04762.x. PubMed PMID: 15142145.
You S, Chen C, Lee W H, Brusko T, Atkinson M, Liu C P. Presence of diabetes-inhibiting, glutamic acid decarboxylase-specific, IL-10-dependent, regulatory T cells in naive nonobese diabetic mice. J Immunol. 2004; 173(11):6777-85. doi: 10.4049/jimmunol.173.11.6777. PubMed PMID: 15557171.
Hellstrom-Lindahl E, Danielsson A, Ponten F, Czernichow P, Korsgren O, Johansson L, Eriksson O. GPR44 is a pancreatic protein restricted to the human beta cell. Acta Diabetol. 2016; 53(3):413-21. doi: 10.1007/s00592-015-0811-3. PubMed PMID: 26467464.
Eriksson O, Johnstrom P, Cselenyi Z, Jahan M, Selvaraju R K, Jensen-Waern M, Takano A, SorhedeWinzell M, Halldin C, Skrtic S, Korsgren O. In Vivo Visualization of beta-Cells by Targeting of GPR44. Diabetes. 2018; 67(2):182-92. doi: 10.2337/dbl7-0764. PubMed PMID: 29208633.
Tang Q, Adams J Y, Penaranda C, Melli K, Piaggio E, Sgouroudis E, Piccirillo C A, Salomon B L, Bluestone J A. Central role of defective interleukin-2 production in the triggering of islet autoimmune destruction. Immunity. 2008; 28(5):687-97. Epub 2008/05/13. doi: 10.1016/j.immuni.2008.03.016. PubMed PMID: 18468463; PMCID: PMC2394854.
Samy E T, Wheeler K M, Roper R J, Teuscher C, Tung K S. Cutting edge: Autoimmune disease in day 3 thymectomized mice is actively controlled by endogenous disease-specific regulatory T cells. J Immunol. 2008; 180(7):4366-70. doi: 10.4049/jimmunol.180.7.4366. PubMed PMID: 18354156.
Du W, Shen Y W, Lee W H, Wang D, Paz S, Kandeel F, Liu C P. Foxp3+ Treg expanded from patients with established diabetes reduce Helios expression while retaining normal function compared to healthy individuals. PLoS One. 2013; 8 (2): e56209. doi: 10.1371/journal.pone.0056209. PubMed PMID: 23409157; PMCID: PMC3569420.
Wang D, Qin H, Du W, Shen Y W, Lee W H, Riggs A D, Liu C P. Inhibition of S-phase kinase-associated protein 2 (Skp2) reprograms and converts diabetogenic T cells to Foxp3+ regulatory T cells. Proc Natl Acad Sci USA. 2012; 109(24):9493-8. doi: 10.1073/pnas.1207293109. PubMed PMID: 22645357; PMCID: PMC3386130.
Domogalla M P, Rostan P V, Raker V K, Steinbrink K. Tolerance through Education: How Tolerogenic Dendritic Cells Shape Immunity. Front Immunol. 2017; 8:1764. doi: 10.3389/fimmu.2017.01764. PubMed PMID: 29375543; PMCID: PMC5770648.
Obregon C, Kumar R, Pascual M A, Vassalli G, Golshayan D. Update on Dendritic Cell-Induced Immunological and Clinical Tolerance. Front Immunol. 2017; 8:1514. doi: 10.3389/fimmu.2017.01514. PubMed PMID: 29250057; PMCID: PMC5715373.
Hull C M, Nickolay L E, Estorninho M, Richardson M W, Riley J L, Peakman M, Maher J, Tree T I. Generation of human islet-specific regulatory T cells by TCR gene transfer. J Autoimmun. 2017; 79:63-73. doi: 10.1016/j.jaut.2017.01.001. PubMed PMID: 28117148.
Martinez N R, Augstein P, Moustakas A K, Papadopoulos G K, Gregori S, Adorini L, Jackson D C, Harrison L C. Disabling an integral CTL epitope allows suppression of autoimmune diabetes by intranasal proinsulin peptide. J Clin Invest. 2003; 111(9):1365-71. doi: 10.1172/JCI17166. PubMed PMID: 12727928; PMCID: PMC154446.
Cianciaruso C, Phelps E A, Pasquier M, Hamelin R, Demurtas D, Alibashe Ahmed M, Piemonti L, Hirosue S, Swartz M A, De Palma M, Hubbell J A, Baekkeskov S. Primary Human and Rat beta-Cells Release the Intracellular Autoantigens GAD65, IA-2, and Proinsulin in Exosomes Together With Cytokine-Induced Enhancers of Immunity. Diabetes. 2017; 66(2):460-73. doi: 10.2337/dbl6-0671. PubMed PMID: 27872147.
Ohkura N, Kitagawa Y, Sakaguchi S. Development and maintenance of regulatory T cells. Immunity. 2013; 38(3):414-23. Epub 2013/03/26. doi: 10.1016/j.immuni.2013.03.002. PubMed PMID: 23521883.
Fransson M, Piras E, Burman J, Nilsson B, Essand M, Lu B, Harris R A, Magnusson P U, Brittebo E, Loskog A S. CAR/FoxP3-engineered T regulatory cells target the CNS and suppress EAE upon intranasal delivery. J Neuroinflammation. 2012; 9:112. doi: 10.1186/1742-2094-9-112. PubMed PMID: 22647574; PMCID: PMC3403996.
Elinav E, Adam N, Waks T, Eshhar Z. Amelioration of colitis by genetically engineered murine regulatory T cells redirected by antigen-specific chimeric receptor. Gastroenterology. 2009; 136(5):1721-31. doi: 10.1053/j.gastro.2009.01.049. PubMed PMID: 19208357.
Elinav E, Waks T, Eshhar Z. Redirection of regulatory T cells with predetermined specificity for the treatment of experimental colitis in mice. Gastroenterology. 2008; 134(7):2014-24. doi: 10.1053/j.gastro.2008.02.060. PubMed PMID: 18424268.
Noyan F, Zimmermann K, Hardtke-Wolenski M, Knoefel A, Schulde E, Geffers R, Hust M, Huehn J, Galla M, Morgan M, Jokuszies A, Manns M P, Jaeckel E. Prevention of Allograft Rejection by Use of Regulatory T Cells With an MHC-Specific Chimeric Antigen Receptor. Am J Transplant. 2017; 17(4):917-30. Epub 2016/12/21. doi: 10.1111/ajt.14175. PubMed PMID: 27997080.
Boardman D A, Philippeos C, Fruhwirth G O, Ibrahim M A, Hannen R F, Cooper D, Marelli-Berg F M, Watt F M, Lechler R I, Maher J, Smyth L A, Lombardi G. Expression of a Chimeric Antigen Receptor Specific for Donor HLA Class I Enhances the Potency of Human Regulatory T Cells in Preventing Human Skin Transplant Rejection. Am J Transplant. 2017; 17(4):931-43. doi: 10.1111/ajt.14185. PubMed PMID: 28027623.
Tenspolde M, Zimmermann K, Weber L C, Hapke M, Lieber M, Dywicki J, Frenzel A, Hust M, Galla M, Buitrago-Molina L E, Manns M P, Jaeckel E, Hardtke-Wolenski M. Regulatory T cells engineered with a novel insulin-specific chimeric antigen receptor as a candidate immunotherapy for type 1 diabetes. J Autoimmun. 2019; 103:102289. doi: 10.1016/j.jaut.2019.05.017. PubMed PMID: 31176558.
Brown C E, Alizadeh D, Starr R, Weng L, Wagner J R, Naranjo A, Ostberg J R, Blanchard M S, Kilpatrick J, Simpson J, Kurien A, Priceman S J, Wang X, Harshbarger T L, D'Apuzzo M, Ressler J A, Jensen M C, Barish M E, Chen M, Portnow J, Forman S J, Badie B. Regression of Glioblastoma after Chimeric Antigen Receptor T-Cell Therapy. N Engl J Med. 2016; 375(26):25619. doi: 10.1056/NEJMoa1610497. PubMed PMID: 28029927; PMCID: PMC5390684.
Weist M R, Starr R, Aguilar B, Chea J, Miles J K, Poku E, Gerdts E, Yang X, Priceman S J, Forman S J, Colcher D, Brown C E, Shively J E. PET of Adoptively Transferred Chimeric Antigen Receptor T Cells with (89)Zr-Oxine. J Nucl Med. 2018; 59(10):1531-7. doi: 10.2967/jnumed.117.206714. PubMed PMID: 29728514; PMCID: PMC6167529.
King J D, Ma Y, Kuo Y C, Bzymek K P, Goodstein L H, Meyer K, Moore R E, Crow D, Colcher D M, Singh G, Horne D A, Williams J C. Template-Catalyzed, Disulfide Conjugation of Monoclonal Antibodies Using a Natural Amino Acid Tag. Bioconjug Chem. 2018; 29(6):2074-81. doi: 10.1021/acs.bioconjchem.8b00284. PubMed PMID: 29763554.

What is claimed is:

1. A method of generating a reprogrammed T regulatory (Treg) cell, the method comprising:
(a) contacting a dendritic cell (DC) with interleukin-10 (IL-10), thereby producing a tolerogenic dendritic cell (tolDC);
(b) contacting said tolDC with an islet extracellular vesicle (EV), thereby producing an antigen-loaded tolDC; and
(c) contacting a Treg cell with said antigen-loaded tolDC, thereby producing said reprogrammed Treg cell.

2. The method of claim 1, wherein said Treg cell is obtained from a subject with Type 1 diabetes.

3. The method of claim 1, wherein said DC is obtained from a subject with Type 1 diabetes.

4. The method of claim 1, wherein said Treg cell and said DC are obtained from the same subject.

5. The method of claim 1, wherein said islet EV is obtained from a donor who does not have Type 1 diabetes.

6. The method of claim 1, wherein step (a) further comprises expanding said tolDC.

7. The method of claim 1, wherein step (b) further comprises expanding said antigen-loaded tolDC.

8. The method of claim 1, wherein step (c) further comprises expanding said reprogrammed Treg cell.

9. The method of claim 8, wherein said reprogrammed Treg cell is expanded in the presence of IL-2.

10. The method of claim 1, wherein said reprogrammed Treg cell express higher levels of an immunosuppressive cytokine compared to a polyclonal Treg cell.

11. The method of claim 10, wherein said immunosuppressive cytokine is IL-10 and/or tumor growth factor-β (TGF-β).

12. The method of claim 1, wherein said reprogrammed Treg cell expresses lower levels of a proinflammatory cytokine compared to a polyclonal Treg.

13. The method of claim 12, wherein said proinflammatory cytokine is interferon-γ (IFN-γ), tumor necrosis factor-α (TNF-α), interleukin-4 (IL-4), and/or interleukin-6 (IL-6).

14. The method of claim 1, wherein said reprogrammed Treg cell increases suppression of CD4+ and CD8+ T cell proliferation compared to a polyclonal Treg cell.

15. A reprogrammed Treg cell made by the method of claim 1.

16. The reprogrammed Treg cell of claim 15, wherein said reprogrammed Treg cell express higher levels of an immunosuppressive cytokine compared to a polyclonal Treg cell.

17. The reprogrammed Treg cell of claim 16, wherein said immunosuppressive cytokine is IL-10 and/or tumor growth factor-β (TGF-β).

18. The reprogrammed Treg cell of claim 15, wherein said reprogrammed Treg cell expresses lower levels of a proinflammatory cytokine compared to a polyclonal Treg cell.

19. The reprogrammed Treg cell of claim 18, wherein said proinflammatory cytokine is interferon-γ (IFN-γ), tumor necrosis factor-α (TNF-α), interleukin-4 (IL-4), and/or interleukin-6 (IL-6).

20. The reprogrammed Treg cell of claim 15, wherein said reprogrammed Treg cell targets a protein expressed on the surface of a pancreatic beta cell.

21. The reprogrammed Treg cell of claim 20, wherein said protein is Sodium/potassium-transporting ATPase subunit gamma (FXYD2), NTPDase3, Glucagon-like peptide-1 receptor (GLP-1R) or GPR44.

22. The reprogrammed Treg cell of claim 15, comprising a chimeric antigen receptor (CAR).

23. The reprogrammed Treg cell of claim 22, wherein said CAR comprises:
(i) an antibody region comprising a FXYD2, NTPDase3, GLP-1R or GPR44 binding domain; and
(ii) a transmembrane binding domain.

24. The reprogrammed Treg cell of claim 15, further comprising a detectable moiety.

25. A method of treating Type 1 diabetes in a subject in need thereof, said method comprising administering to said subject a therapeutically effective amount of the reprogrammed Treg cell of claim 15.

26. A method of treating or preventing Type 1 diabetes in a subject in need thereof, the method comprising:
(a) contacting a tolerogenic dendritic cell (tolDC) with an islet extracellular vesicle (EV), thereby producing an antigen-loaded tolDC;
(b) contacting a T regulatory (Treg) cell with said antigen-loaded tolDC, thereby generating a reprogrammed Treg cell; and
(c) administering a therapeutically effective amount of said reprogrammed Treg cell to said subject, thereby treating or preventing Type 1 diabetes in the subject.

27. The method of claim 26, wherein said tolDC is produced by contacting a dendritic cell (DC) with interleukin-10 (IL-10).

28. The method of claim 26, wherein said Treg cell is obtained from a subject with Type 1 diabetes.

29. The method of claim 26, wherein said DC is obtained from a subject with Type 1 diabetes.

30. The method of claim 26, wherein said Treg cell and said CD are obtained from the same subject.

31. The method of claim 26, wherein said islet EV is obtained from a donor who does not have Type 1 diabetes.

32. The method of claim 26, wherein step (a) further comprises expanding said tolDC.

33. The method of claim 26, wherein step (a) further comprises expanding said antigen-loaded tolDC.

34. The method of claim 26, wherein step (b) further comprises expanding said reprogrammed Treg cell.

35. The method of claim 34, wherein said reprogrammed Treg cell is expanded in the presence of IL-2.

36. The method of claim 26, wherein said reprogrammed Treg express higher levels of an immunosuppressive cytokine compared to a polyclonal Treg.

37. The method of claim 36, wherein said immunosuppressive cytokine is IL-10 and/or tumor growth factor-β (TGF-β).

38. The method of claim 26, wherein said reprogrammed Treg cell expresses lower levels of a proinflammatory cytokine compared to a polyclonal Treg.

39. The method of claim 38, wherein said proinflammatory cytokine is interferon-γ (IFN-γ), tumor necrosis factor-α (TNF-α), interleukin-4 (IL-4), and/or interleukin-6 (IL-6).
